(19) United States
(12) Patent Application Publication
(10) Pub. No.: US 2025/0339447 A1
(43) Pub. Date: Nov. 6, 2025

(54) 19-NOR C3,3-DISUBSTITUTED C21-N-PYRAZOLYL STEROID AND METHODS OF USE THEREOF

(71) Applicant: Sage Therapeutics, Inc., Cambridge, MA (US)

(72) Inventors: Stephen Jay Kanes, Villanova, PA (US); James J. Doherty, Bedford, MA (US); Handan Gunduz-Bruce, Lexington, MA (US); Jeffrey M. Jonas, Vero Beach, FL (US); Robert Alfonso Lasser, Brookline, MA (US)

(21) Appl. No.: 18/978,431

(22) Filed: Dec. 12, 2024

Related U.S. Application Data
(63) Continuation of application No. 17/843,031, filed on Jun. 17, 2022, now abandoned.
Application No. 17/843,031 is a continuation of application No. 16/704,800, filed on Dec. 5, 2019, now abandoned.
Application No. 16/704,800 is a continuation-in-part of application No. PCT/US2019/036848, filed on Jun. 12, 2019.
(60) Provisional application No. 62/684,155, filed on Jun. 12, 2018.
(60) Provisional application No. 62/789,329, filed on Jan. 7, 2019.
(60) Provisional application No. 62/841,645, filed on May 1, 2019.

(51) Int. Cl. A61K 31/58; A61P 25/24; A61P 25/28

(52) U.S. Cl. CPC A61K 31/58; A61P 25/24; A61P 25/28

(57) ABSTRACT

Provided herein are methods for treating depression, such as postpartum depression or major depressive disorder, in a subject in need thereof, comprising administering to the subject an effective amount of Compound 1 or a pharmaceutically acceptable salt thereof:

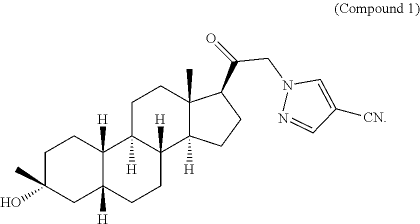

### CROSS REFERENCE TO RELATED APPLICATIONS

This application is a continuation-in-part of PCT/US2019/036848, which is incorporated herein by reference and claims priority to and the benefit of U.S. Provisional Patent Application Nos. 62/684,155 filed Jun. 12, 2018, 62/789,329 filed Jan. 7, 2019, and 62/841,645 filed May 1, 2019.

### FIELD OF THE INVENTION

The present invention generally relates to methods of treating depression such as postpartum depression and major depressive disorder by administering Compound 1 as described herein.

### BACKGROUND

GABA, γ-aminobutyric acid, has a profound influence on overall brain excitability because up to 40% of the neurons in the brain utilize GABA as a neurotransmitter. GABA interacts with its recognition site on the GRC (GABA receptor complex) to facilitate the flow of chloride ions down an electrochemical gradient of the GRC into the cell. An intracellular increase in the levels of this anion causes hyperpolarization of the transmembrane potential, rendering the neuron less susceptible to excitatory inputs (i.e., reduced neuron excitability). In other words, the higher the chloride ion concentration in the neuron, the lower the brain excitability (the level of arousal). It is well-documented that the GRC is responsible for the mediation of anxiety, seizure activity, and sedation. Thus, GABA and drugs that act like GABA (e.g., the therapeutically useful barbiturates and benzodiazepines (BZs), such as Valium®) produce their therapeutically useful effects by interacting with specific regulatory sites on the GRC.

Accumulated evidence has indicated that the GRC contains a distinct site for neuroactive steroids (Lan, N. C. et al., Neuwchem. Res. 16:347-356 (1991)). Neuroactive steroids can occur endogenously. The most potent endogenous neuroactive steroids are 3α-hydroxy-5-reduced pregnan-20-one and 3α-21-dihydroxy-5-reduced pregnan-20-one, metabolites of hormonal steroids progesterone and deoxycorticosterone, respectively. The ability of these steroid metabolites to alter brain excitability was recognized in 1986 (Majewska, M. D. et al., Science 232:1004-1007 (1986); Harrison, N. L. et al., J. Pharmacol. Exp. Ther. 241:346-353 (1987)).

Compound 1, a neuroactive steroid described herein, has been shown to be a positive allosteric modulator of GABA_{A} receptors that targets synaptic and extrasynaptic GABA_{A} receptors. As a positive allosteric modulator of GABA_{A} receptors, Compound 1 serves as a therapeutic agent to treat CNS related disorders, e.g., depression, e.g., postpartum depression and major depressive disorder. Current treatments for CNS related disorders typically requires extended, sometimes chronic, treatment, and patient compliance can be a major problem. Those who suffer from CNS related disorders would benefit significantly from a new treatment regime which is effective, is easy to administer and/or requires fewer administrations and avoids or minimizes side effects.

### SUMMARY

Provided herein are methods of treating depression in a subject, the method comprising administering to the subject a therapeutically effective amount of Compound 1

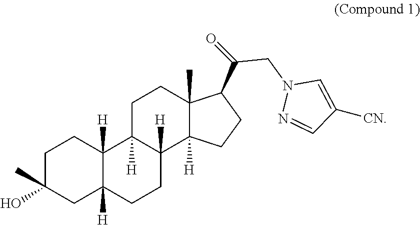

or a pharmaceutically acceptable salt thereof. In further embodiments, Compound 1 is administered using an episodic dosing regimen. In the methods described herein, Compound 1 or a pharmaceutically acceptable salt thereof can be administered for a specified period of time, e.g., 14 days. Following such specified periods of time, the subject is not dosed with the compound for another specified period of time, e.g., at least a period of e.g., about 2 weeks, about 3 weeks, about 4 weeks, about 5 weeks, or about 6 weeks.

In some aspects, provided herein is an episodic dosing regimen comprising administering Compound 1 to a subject in need thereof. In some embodiments, about 10 mg, about 15 mg, about 20 mg, about 25 mg, about 30 mg, about 40 mg, or about 45 mg of Compound 1 is administered to a subject in need thereof. In some embodiments, about 10 mg, about 15 mg, about 20 mg, about 25 mg or about 30 mg of Compound 1 is administered to the subject in need thereof. In some embodiments, Compound 1 is administered to a subject in need thereof once a day for a plurality of weeks, e.g., about 2 weeks to about 6 weeks, e.g., about 2 weeks to about 4 weeks, e.g., about 2 weeks. In some embodiments, about 10 mg, about 15 mg, about 20 mg, about 25 mg or about 30 mg of Compound 1 is administered to a subject in need thereof once a day for a plurality of weeks. In some embodiments, about 10 mg, about 15 mg, about 20 mg, about 25 mg, about 30 mg, about 40 mg, or about 45 mg of Compound 1 is administered to a subject in need thereof once a day for a plurality of weeks.

In a preferred embodiment, Compound 1 is administered using an episodic dosing regimen, where the episodic dosing regimen occurs for about 2 weeks to about 6 weeks. In a more preferred embodiment, the episodic dosing regimen occurs for about 2 weeks to about 4 weeks. In an even more preferred embodiment, the episodic dosing regimen occurs for about 2 weeks (or about 14 days). In another embodiment, the episodic dosing regimen has a duration of 2 weeks, i.e., 14 days.

In some aspects, provided herein is an episodic dosing regimen for treating depression comprising administrating Compound 1 to a subject in need thereof. In some embodiments, about 10 mg, about 15 mg, about 20 mg, about 25 mg or about 30 mg of Compound 1 is administered to the subject. In some embodiments, about 10 mg, about 15 mg, about 20 mg, about 25 mg, about 30 mg, about 40 mg, or about 45 mg of Compound 1 is administered to the subject. In some embodiments, Compound 1 is administered to the subject once a day for a plurality of weeks. In some embodiments, about 10 mg, about 15 mg, about 20 mg, about 25 mg or about 30 mg of Compound 1 is administered to the subject once a day for a plurality of weeks. In some embodiments, about 10 mg, about 15 mg, about 20 mg, about 25 mg, about 30 mg, about 40 mg, or about 45 mg of Compound 1 is administered to the subject once a day for a plurality of weeks. In a preferred embodiment, the episodic dosing regimen occurs for about 2 weeks to about 6 weeks. In a more preferred embodiment, the episodic dosing regimen occurs for about 2 weeks to about 4 weeks. In an even more preferred embodiment, the episodic dosing regimen occurs for about 2 weeks. In an even more preferred embodiment, the episodic dosing regimen occurs for about 14 days. In another embodiment, the episodic dosing regimen has a duration of 2 weeks, i.e., 14 days. In an even more preferred embodiment, the episodic dosing regimen occurs for about 2 weeks (or about 14 days), wherein the subject is administered about 30 mg of Compound 1 once a day during the 2 week period (or about 14 days). If the subject does not tolerate administration of about 30 mg of Compound 1 once a day, the subject is administered about 20 mg of Compound 1 once a day.

In some embodiments, the subject exhibits a response to the episodic dosing regimen, wherein the response is indicated by greater than or equal to about 50% reduction in HAM-D score from baseline. In some embodiments, the response is indicated by a remission of depression symptoms.

In some embodiments, the subject is evaluated for recurrence, i.e., reappearance of depression symptoms. In some embodiments, the method of treating the subject comprises a plurality of episodic dosing regimens. In some embodiments, after completion of an episodic dosing regimen, a subsequent episodic dosing regimen is administered with the reappearance of depression symptoms. In some embodiments, the episodic dosing regimens are spaced apart by at least a 6 week interval. In some embodiments, the episodic dosing regimens are spaced apart by 6 weeks. In some embodiments, the episodic dosing regimens are spaced apart by 7 weeks. In some embodiments, the episodic dosing regimens are spaced apart by 8 weeks.

In some aspects, provided herein is an episodic dosing regimen for treating major depressive disorder, bipolar depression, anxiety, or postpartum depression, comprising dosing of Compound 1 to a subject in need thereof. In some embodiments, the major depressive disorder is moderate major depressive disorder. In some embodiments, the major depressive disorder is severe major depressive disorder. In some embodiments, about 10 mg, about 15 mg, about 20 mg, about 25 mg or about 30 mg of Compound 1 is administered to the subject. In some embodiments, about 10 mg, about 15 mg, about 20 mg, about 25 mg, about 30 mg, about 40 mg, or about 45 mg of Compound 1 is administered to the subject. In some embodiments, Compound 1 is administered once a day for a plurality of weeks, e.g., about 2 weeks to about 6 weeks, e.g., about 2 weeks to about 4 weeks, e.g., about 2 weeks to treat treating major depressive disorder, bipolar depression, anxiety, or postpartum depression. In some embodiments, about 10 mg, about 15 mg, about 20 mg, about 25 mg or about 30 mg of Compound 1 is administered to the subject once a day for a plurality of weeks to treat treating major depressive disorder, bipolar depression, anxiety, or postpartum depression. In some embodiments, about 10 mg, about 15 mg, about 20 mg, about 25 mg, about 30 mg, about 40 mg, or about 45 mg of Compound 1 is administered to the subject once a day for a plurality of weeks to treat treating major depressive disorder, bipolar depression, anxiety, or postpartum depression. In a preferred embodiment, the episodic dosing regimen occurs for about 2 weeks to about 6 weeks. In a more preferred embodiment, the episodic dosing regimen occurs for about 2 weeks to about 4 weeks. In an even more preferred embodiment, the episodic dosing regimen occurs for about 2 weeks, or about 14 days. In another embodiments, the episodic dosing regimen has a duration of 2 weeks.

In some aspects, provided herein is a method of treating depression in a subject in need thereof, comprising administering to said subject an episodic dosing regimen of Compound 1. In some aspects, provided herein is a method of treating postpartum depression in a subject in need thereof, comprising administering to said subject an episodic dosing regimen of 30 mg of Compound 1 once a day for 2 weeks (or about 14 days). If the subject does not tolerate administration of 30 mg of Compound 1 once a day, the subject is administered 20 mg of Compound 1 once a day. In some embodiments, the subject is a human female diagnosed with severe postpartum depression. In some embodiments, the subject has been experiencing a major depressive episode over about a 1-year period. In some embodiments, the subject is between about 18 and about 75 years of age. In some embodiments, the subject is between about 18 and about 65 years of age.

The episodic dosing regimen of the present invention provides the advantage of not being a chronic dosing regimen, unlike current treatments for depression, e.g., major depressive disorder (MDD). Thus, according to the present invention, a pharmaceutically effective amount of Compound 1 is administered in response to each episode of symptom occurrence. This episodic dosing regimen has the advantage of not requiring chronic dosing and thus avoiding numerous detriments of current therapies of depression.

In another aspect, provided herein is a method of treating depression in a subject in need thereof, the method comprising the steps of:
(i) administering once daily to the subject a therapeutically effective amount of a compound having the formula:

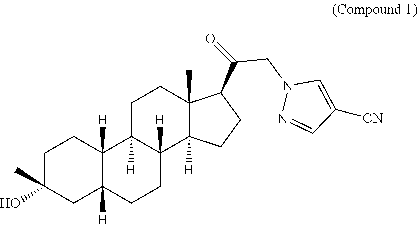

for about two weeks; and
(ii) re-administering once daily to the subject a therapeutically effective amount of Compound 1 for about two weeks in response to a recurrence of depression symptoms;
provided there is an interval between administration of Compound 1 to the subject and re-administration of Compound 1 to the subject.

In some embodiments, Compound 1 is administered to the subject for 2 weeks, i.e., 14 days. In some embodiments, Compound 1 is re-administered to the subject for 2 weeks, i.e., 14 days. In some embodiments, the interval between administration of Compound 1 to the subject and re-administration of Compound 1 to the subject is 2-4 weeks. In some embodiments, the interval is 4 weeks. In some embodiments, the interval is 5 weeks. In some embodiment, the interval is 6 weeks. In some embodiments, the interval between administration of Compound 1 to the subject and re-administration of Compound 1 to the subject is 7 weeks. In some embodiments, the interval between administration of Compound 1 to the subject and re-administration of Compound 1 to the subject is 8 weeks.

In some embodiments, the depression is major depressive disorder (MDD). In some embodiments, the MDD is moderate major depressive disorder. In some embodiments, the MDD is severe major depressive disorder. In some embodiments, the depression is bipolar depression. In some embodiments, the depression is post-partum depression. In some embodiments, the subject has been diagnosed with depression. In some embodiments, the depression is major depressive disorder or bipolar depression. In some embodiments, the subject is a female diagnosed with severe postpartum depression. In some embodiments, the subject has been experiencing a major depressive episode over about a 1-year period. In some embodiments, the subject is between about 18 and about 75 years of age. In some embodiments, the subject is between about 18 and about 65 years of age.

In some embodiments, the method of treating major depressive disorder, bipolar depression, anxiety, or postpartum depression with the administration of Compound 1 improves cognitive function. In other embodiments, the method improves cognitive function in the subject after completing the episodic dosing regimen. In some aspects, the method improves cognitive function in the subject after completing the episodic dosing regimen, wherein the episodic dosing regimen had a duration of about 2 to about 8 weeks. In further aspects, the method improves cognitive function in the subject after completing the episodic dosing regimen, wherein the episodic dosing regimen had a duration of about 2 to about 6 weeks. In other embodiments, the method improves cognitive function in the subject after completing the episodic dosing regimen, wherein the episodic dosing regimen had a duration of about 2 to about 4 weeks. In further embodiments, the method improves cognitive function in the subject after completing the episodic dosing regimen, wherein the episodic dosing regimen had a duration of about 2 weeks or 14 days. In other aspects, the method improves cognitive function in the subject after completing the episodic dosing regimen, wherein the episodic dosing regimen had a duration of 2 weeks.

In some embodiments, the subject is administered about 10 mg of Compound 1. In some embodiments, the subject is administered about 20 mg of Compound 1. In some embodiments, the subject is administered about 30 mg of Compound 1. In some embodiments, the subject is administered about 40 mg of Compound 1. In some embodiments, the subject is administered about 45 mg of Compound 1. In some embodiments, the subject is administered about 10 mg of Compound 1 once a day. In some embodiments, the subject is administered about 20 mg of Compound 1 once a day. In some embodiments, the subject is administered about 30 mg of Compound 1 once a day. In some embodiments, the subject is administered about 40 mg of Compound 1 once a day. In some embodiments, the subject is administered about 45 mg of Compound 1 once a day. In some embodiments, the amount of Compound 1 administered to the subject is reduced in the occurrence of a severe adverse effect. In some embodiments, Compound 1 is administered in the evening. In some embodiments, Compound 1 is administered with food. In some embodiments, Compound 1 is in a capsule. In some embodiments, the method further comprises administration of a second therapeutic agent.

In some aspects, provided herein is a kit comprising a pharmaceutical composition comprising Compound 1 and an instruction set describing a method for using an episodic dosing regimen to treat depression. In some embodiments, the pharmaceutical composition comprises about 10 mg of Compound 1. In some embodiments, the pharmaceutical composition comprises about 15 mg of Compound 1. In some embodiments, the pharmaceutical composition comprises about 20 mg of Compound 1. In some embodiments, the pharmaceutical composition comprises about 25 mg of Compound 1. In some embodiments, the pharmaceutical composition comprises about 30 mg of Compound 1. In some embodiments, the episodic dosing regimen occurs for about 2 weeks to about 6 weeks. In a more preferred embodiment, the episodic dosing regimen occurs for about 2 weeks to about 4 weeks. In an even more preferred embodiment, the episodic dosing regimen occurs for about 2 weeks. In a preferred embodiment, the episodic dosing regimen occurs for 2 weeks. In some embodiments, depression is major depressive disorder, bipolar depression, anxiety, or postpartum depression. In some embodiments, the major depressive disorder is moderate major depressive disorder. In some embodiments, the major depressive disorder is severe major depressive disorder. In some embodiments, the episodic dosing regimen occurs for about 2 weeks (or about 14 days) for the treatment of postpartum depression. In some embodiments, the instruction set is printed on a suitable material. In some embodiments, the individual dosage units are capsules or tablets. In some embodiments, the individual dosage unit is a capsule. In some embodiments, the individual dosage unit is a capsule of size 1, 2, 3, or 4. In some embodiments, the capsule is size 1.

In one aspect, described herein is a method of treating major depressive disorder in a subject in need thereof, comprising administering to the subject a therapeutically effective amount, e.g., about 30 mg, of a compound of the formula:

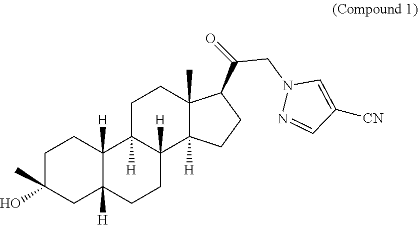

once a day for 14 days.

In some embodiments, the therapeutically effective amount of the compound is 30 mg-40 mg. In some embodiments, the therapeutically effective amount of the compound is 30 mg. In some embodiments, the major depressive disorder is severe major depressive disorder. In some embodiments, the subject exhibits a reduction in depression-related symptoms. In some embodiments, the reduction in depression-related symptoms is characterized by a reduction in HAM-D score from baseline. In some embodiments, the major depressive disorder is characterized by a HAM-D score of at least 22 prior to treatment. In some embodiments, the major depressive disorder is characterized by a HAM-D score of at least 24 prior to treatment. In some embodiments, the major depressive disorder is characterized by a HAM-D score of at least 25 prior to treatment. In some embodiments, the major depressive disorder is characterized by a HAM-D score of at least 26 prior to treatment. In some embodiments, the major depressive disorder is characterized by a MADRS score of at least 32 prior to treatment.

In another aspect, described herein is a method of treating major depressive disorder comprising the steps of: (i) administering once daily to the subject a therapeutically effective amount, e.g., about 30 mg, of a compound of the formula:

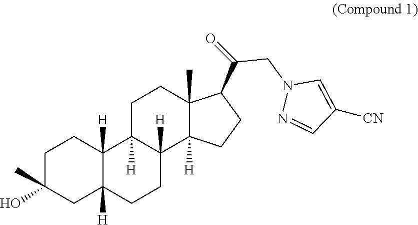

once a day for 14 days; and (ii) re-administering once daily to the subject a therapeutically effective amount, e.g., about 30 mg, of Compound 1 for 15 days in response to a recurrence of depression symptoms, provided there is at least a 6 week interval between administration of Compound 1 to the subject and re-administration of Compound 1 to the subject. In some embodiments, the therapeutically effective amount of the compound is 20 mg-40 mg. In some embodiments, the therapeutically effective amount of the compound is 30 mg. In some embodiments, the major depressive disorder is severe major depressive disorder. In some embodiments, the subject exhibits a reduction in depression-related symptoms. In some embodiments, the reduction in depression-related symptoms is characterized by a reduction in HAM-D score from baseline. In some embodiments, the major depressive disorder is characterized by a HAM-D score of at least 22 prior to treatment. In some embodiments, the major depressive disorder is characterized by a HAM-D score of at least 24 prior to treatment. In some embodiments, the major depressive disorder is characterized by a HAM-D score of at least 25 prior to treatment. In some embodiments, the major depressive disorder is characterized by a HAM-D score of at least 26 prior to treatment. In some embodiments, the major depressive disorder is characterized by a MADRS score of at least 32 prior to treatment.

### BRIEF DESCRIPTION OF THE DRAWINGS

FIG. 1 depicts the LS mean change from baseline in Hamilton Rating Scale for Depression (HAM-D), total score over time by treatment group.

FIG. 2 depicts a forest plot of the subgroup analysis for primary endpoint at day 15.

FIG. 3 depicts a bar chart of Hamilton Rating Scale for Depression (HAM-D) remission by time point and treatment group.

FIG. 4 depicts a bar chart of Hamilton Rating Scale for Depression (HAM-D) remission by time point and treatment group.

FIG. 5 depicts change from base in Montgomery-Asberg Depression Rating Scale (MADRS), total score over time by treatment group.

FIG. 6 depicts change from baseline in Hamilton Anxiety Rating Scale (HAM-A), total score over time by treatment group.

FIG. 7 depicts a bar chart of clinical global impression (CGI) Improvement Response by Time Point and Treatment Group.

FIG. 8 depicts an exemplary study design for treating MDD with Compound 1.

FIG. 9 depicts an exemplary study design for treating MDD with Compound 1.

FIG. 10 depicts exemplary HAM-D total score LS mean changes from baseline to Day 15 in patients in a Phase 3 study of Compound 1 in Major Depressive Disorder.

FIG. 11 depicts exemplary post-hoc analyses in the primary analysis, in patients with measurable drug concentrations, in patients with HAM-D≥24, and in patients with measurable drug concentrations and HAM-D≥24, and including the primary outcome at Day 15, of a Phase 3 study of Compound 1 in Major Depressive Disorder.

FIG. 12 depicts exemplary post-hoc analyses in the primary analysis, in patients with measurable drug concentrations, in patients with HAM-D≥24, and in patients with measurable drug concentrations and HAM-D≥24, and including the primary outcome at Day 15 and double-blind follow up periods and follow-periods/interim analyses, of a Phase 3 study of Compound 1 in Major Depressive Disorder.

### DETAILED DESCRIPTION

As generally described herein, the present invention provides compounds and compositions useful for treating depression such as postpartum depression and major depressive disorder.

### Definitions

As used herein, the term “unit dosage form” is defined to refer to the form in which Compound 1 is administered to the subject. Specifically, the unit dosage form can be, for example, a pill, capsule, or tablet. Preferably, the unit dosage form is a capsule. The typical amount of Compound 1 in a unit dosage form useful in the invention is about 10 mg to about 100 mg, preferably about 10 mg to about 50 mg (e.g., about, 10, about 15, about 20, about 25 mg or about 30 mg).

In a preferred embodiment of the invention, the unit dosage form comprises about 30 mg of Compound 1 and is in the form of a capsule. In another preferred embodiment of the invention, the unit dosage form comprises about 45 mg Compound 1 and is in the form of a capsule. In another preferred embodiment of the invention, the unit dosage form comprises about 20 mg of Compound 1 and is in the form of a capsule. In another preferred embodiment of the invention, the unit dosage form comprises about 10 mg of Compound 1 and is in the form of a capsule. In another preferred embodiment of the invention, the unit dosage form comprises about 15 mg of Compound 1 and is in the form of a capsule. In another preferred embodiment of the invention, the unit dosage form comprises about 25 mg of Compound 1 and is in the form of a capsule. Preferably, capsules which comprise about 30 mg or 45 mg of Compound 1, is administered to a subject once per day. In some embodiments, three capsules together comprise the 30 mg of Compound 1. In some embodiments, three capsules together comprises the 45 mg of Compound 1.

As used herein, “solid dosage form” means a pharmaceutical dose(s) in solid form, e.g. tablets, capsules, granules, powders, sachets, reconstitutable powders, dry powder inhalers and chewables.

Where the use of the term “about” is before a quantitative value, the present teachings also include the specific quantitative value itself, unless specifically stated otherwise. As used herein, the term “about” refers to a ±10% variation from the nominal value unless otherwise indicated or inferred.

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

A “subject” is a human (i.e., a male or female of any age group, e.g., a pediatric subject (e.g., infant, child, adolescent) or adult subject (e.g., young adult, middle-aged adult or senior adult)).

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, the “effective amount” of a compound refers to an amount sufficient to elicit the desired biological response, e.g., to treat a CNS-related disorder, e.g., a disorder as described herein (e.g., tremor (e.g., essential tremor); depression (e.g., postpartum depression); or an anxiety disorder). As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, an “episodic dosing regimen” is a dosing regimen wherein a compound or a composition comprising a compound is administered to a subject for a finite period of time in response to the diagnosis of a disorder or symptom thereof, e.g., a diagnosis or symptom of depression, an episode of major depressive disorder, bipolar depression, anxiety, or postpartum depression. In some embodiments, the major depressive disorder is moderate major depressive disorder. In some embodiments, the major depressive disorder is severe major depressive disorder. In some embodiments, the compound is formulated as individual dosage units, each unit comprising Compound 1 and one or more suitable pharmaceutical excipient. In some embodiments, the episodic dosing regimen has a duration of a plurality of weeks, e.g. about 8 weeks. In contrast with chronic administration as defined herein, episodic dosing of a compound occurs over a finite period of time, e.g., from about 2 weeks to about 8 weeks, in response to a diagnosis or recurrence of a disorder, e.g., depression, or a symptom thereof. In some embodiments, episodic dosing occurs once per day across a plurality of weeks, e.g., from about 2 weeks to about 6 weeks. In one embodiment, the episodic dosing has a duration of two weeks. In some embodiments, more than one episodic dosing regimen is administered to the subject, e.g., two or more episodic regimens throughout the subject's life.

In some embodiments, administering Compound 1 improves cognitive function. In some embodiments, the cognitive function refers to a collection of mental tasks and functions, including but not limited to: memory (e.g., semantic, episodic, procedural, priming, or working); orientation; language; problem solving; visual perception, construction, and integration; planning; organizational skills; selective attention; inhibitory control; and ability to mentally manipulate information. In one embodiment, the cognitive function is one or more selected from the group consisting of memory (e.g., semantic, episodic, procedural, priming, or working); orientation; language; problem solving; visual perception, construction, and integration; planning; organizational skills; selective attention; inhibitory control; and ability to mentally manipulate information. Measures of cognitive functioning include assessment tools designed to measure, for example: (a) general intelligence, (b) nonverbal intelligence, (c) achievement, (d) attention/executive functioning, (e) memory and learning, (f) visual-motor and motor functioning and (g) language.

Any change in cognitive function, for example, over time or through treatment, can be monitored by using one or more of these well-established tests at two or more time points and comparing the results. The phrase “improves cognitive function”, as referred to herein, means a positive change in the ability of the subject to perform a symbolic operation, for example, to perceive, remember, create a mental image, have clarity of thought, be aware, to reason, think or judge. The positive change can be measured using any of the aforementioned tests on two or more occasions, for example, a first occasion to measure baseline cognitive function and a second occasion to measure cognitive function following a period of time (in which treatment may have been administered). Such assessment tools are well-known in the art and include, for example, those assessment tools as described in Example 4 herein.

### Pharmaceutical Compositions

In one aspect, the disclosure provides a pharmaceutical composition comprising a compound of the present invention (also referred to as the “active ingredient”), for example Compound 1, and a pharmaceutically acceptable excipient. In certain embodiments, the pharmaceutical composition comprises an effective amount of the active ingredient. In certain embodiments, the pharmaceutical composition comprises a therapeutically effective amount of the active ingredient. In certain embodiments, the pharmaceutical composition comprises a prophylactically effective amount of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In preferred embodiments, Compound 1 is administering to a subject orally.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

When used to prevent the onset of a CNS-disorder, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present invention can also be administered in sustained release forms or from sustained release drug delivery systems. A description of representative sustained release materials can be found in Remington's Pharmaceutical Sciences.

The present invention also relates to the pharmaceutically acceptable acid addition salt of a compound of the present invention. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

### Methods of Use

Described herein are methods of treating depression, such as postpartum depression, major depressive disorder, or anxiety, such as generalized anxiety disorder, in a subject, the method comprising administering to the subject an effective amount of Compound 1 or a pharmaceutically acceptable salt thereof. The severity of disorders treated by the methods described herein, e.g. moderate or severe, can be characterized by methods known to one of skill in the art. These methods can include, but are not limited to, Hamilton Depression Score (HAM-D), Hamilton Anxiety Score (HAM-A), Montgomery-Asberg Depression Rating Scale (MADRS), and Clinical Global Impression-Improvement Scale (CGI).

Thus, in one aspect, provided herein is a method of treating depression, such as postpartum depression or major depressive disorder, or anxiety, such as generalized anxiety disorder, in a subject, the method comprising administering to the subject a therapeutically effective amount of Compound 1 or a pharmaceutically acceptable salt thereof. In some embodiments, the method comprises administering to the subject a therapeutically effective amount of Compound 1. In some embodiments, the subject is between and including the ages of 18 and 64. In some embodiments, the subject is between and including the ages of 18 and 75. In some embodiments, Compound 1 is administered with food. In some embodiments, the therapeutically effective amount of Compound 1 is 20 mg. In some embodiments, the therapeutically effective amount of Compound 1 is 10 mg. In some embodiments, the therapeutically effective amount of Compound 1 is 15 mg. In some embodiments, the therapeutically effective amount of Compound 1 is 25 mg. In some embodiments, the therapeutically effective amount of Compound 1 is about 30 mg. In some embodiments, the therapeutically effective amount of Compound 1 is about 40 mg. In some embodiments, the therapeutically effective amount of Compound 1 is about 45 mg. In some embodiments, Compound 1 is administered in one or more capsules. In some embodiments, the therapeutically effective amount is administered across three capsules. In some embodiments, the subject does not have an underlying condition. In some embodiments, the subject has an underlying condition.

In some aspects, provided herein are methods for treating a subject with depression or anxiety, said method comprising administering to said subject a pharmaceutical composition comprising Compound 1 using an episodic dosing regimen effective to treat depression in said subject. In some aspects, the dosing regimen is for a duration of about 2 weeks to about 8 weeks. In some other aspects, the dosing regimen is for a duration of about 2 weeks to about 6 weeks. In some other aspects, the dosing regimen is for a duration of about 2 weeks to about 4 weeks. In some other aspects, the dosing regimen is for a duration of about 2 weeks. In some other aspects, the dosing regimen is for a duration of about 2 weeks, or about 14 days.

In some embodiments, about 10 mg of Compound 1 is administered to the subject once a day for a plurality of weeks. In some embodiments, about 15 mg of Compound 1 is administered to the subject once a day for a plurality of weeks. In some embodiments, about 20 mg of Compound 1 is administered to the subject once a day for a plurality of weeks. In some embodiments about 25 mg of Compound 1 is administered to the subject once a day for a plurality of weeks. In some embodiments, 30 mg of Compound 1 is administered to a subject once a day. In some embodiments, 30 mg of Compound 1 is administered to a subject once a day, and if the subject does not tolerate 30 mg of Compound 1, the subject is administered 20 mg of Compound 1 once a day. In some embodiments, 30 mg of Compound 1 is administered to a subject once a day for about two weeks. In some embodiments, 30 mg of Compound 1 is administered to a subject once a day for about two weeks (or about 14 days), and if the subject does not tolerate 30 mg of Compound 1, the subject is administered 20 mg of Compound 1 once a day for about two weeks (or about 14 days). In some embodiments, 30 mg of Compound 1 is administered to a subject once a day for two weeks, and if the subject does not tolerate 30 mg of Compound 1, the subject is administered 20 mg of Compound 1 once a day for two weeks. In some embodiments, 30 mg of Compound 1 is administered to a subject once a day for at least two weeks (or about 14 days). In some embodiments, 30 mg of Compound 1 is administered to a subject once a day for two weeks (or about 14 days), and if the subject does not tolerate 30 mg of Compound 1, the subject is administered 20 mg of Compound 1 once a day for at least two weeks (or about 14 days). In some embodiments, 30 mg of Compound 1 is administered to a subject once a day for two weeks.

In some aspects, the method comprises an episodic dosing regimen, wherein the method comprises administering Compound 1 to a subject concurrent with an episode of a disorder being treated, e.g., an episode of major depressive disorder, bipolar depression, anxiety, including generalized anxiety disorder, or postpartum depression. an episode of major depressive disorder, bipolar depression, anxiety, or postpartum depression. In some embodiments, the major depressive disorder is moderate major depressive disorder. In some embodiments, the major depressive disorder is severe major depressive disorder. In some embodiments, the major depressive disorder is moderate major depressive disorder. In some embodiments, the major depressive disorder is severe major depressive disorder. In some embodiment, the anxiety is generalized anxiety disorder.

In some embodiments, the subject exhibits a response to the episodic dosing regimen, wherein the response is indicated by greater than or equal to about 50% reduction in HAM-D score from baseline.

In some embodiments, the subject is evaluated for recurrence, of depression symptoms. In some embodiments, the method of treating comprises a plurality of episodic dosing regimen. In some embodiments, the episodic dosing regimens are spaced apart by at least a 6 week interval. In some embodiments, the episodic dosing regimens are spaced apart by 6 weeks. In some embodiments, the episodic dosing regimens are spaced apart by 7 weeks. In some embodiments, the episodic dosing regimens are spaced apart by 8 weeks.

In some aspects, provided herein is a method of treating postpartum depression in a subject in need thereof, comprising the steps of administering to said subject an episodic dosing regimen of 30 mg of Compound 1 once a day for about 2 weeks (or about 14 days) and if the subject does not tolerate administration of 30 mg of Compound 1 once a day, the subject is administered 20 mg of Compound 1 once a day.

In some embodiments, the method provides therapeutic effect (e.g., as measured by reduction in Hamilton Depression Score (HAM-D)) within about 45, about 21, about 15, about 8, or about 3 days. In some embodiments, the therapeutic effect is a decrease from baseline in HAM-D score at the end of a treatment period (e.g., about 45, about 21, about 15, about 8, or about 3 days after beginning administration or episodic dosing). In some embodiments, the decrease from baseline in HAM-D score is from severe (e.g., HAM-D score of 24 or greater; or a score of 26 or greater) to symptom-free, i.e. remission of depression (e.g., HAM-D score of 7 or lower). In some embodiments, the decrease from baseline in HAM-D score is from severe (e.g., HAM-D score of 24 or greater; or a score of 26 or greater) to normal or mild depression (e.g., HAM-D score of 7 or lower; or HAM-D score of 18-13).

In some embodiments, the method provides therapeutic effect (e.g., as measured by reduction in Montgomery-Asberg Depression Rating Scale (MADRS)) within about 45, about 21, about 15, about 8, or about 3 days or less. The Montgomery-Åsberg Depression Rating Scale (MADRS) is a ten-item diagnostic questionnaire (regarding apparent sadness, reported sadness, inner tension, reduced sleep, reduced appetite, concentration difficulties, lassitude, inability to feel, pessimistic thoughts, and suicidal thoughts) which psychiatrists use to measure the severity of depressive episodes in patients with mood disorders. 0-6 indicates normal/symptom absent; 7-19 indicates mild depression; 20-34 indicates moderate depression; and >34 indicates severe depression. In some embodiments, the therapeutic effect is a decrease from baseline in MADRS score at the end of a treatment period (e.g., about 45, about 21, about 15, about 8, or about 3 days or less). In some embodiments, the decrease from baseline in MADRS score is from severe (e.g., MADRS score of 30 or greater) to symptom-free (e.g., MADRS score of 20 or lower). For example, the mean change from baseline in MADRS total score from treatment with a compound described herein is about −15, −20, −25, −30, while the mean change from baseline in MADRS total score from treatment with placebo is about −15, −10, −5.

In some embodiments, the method provides therapeutic effect (e.g., as measured by reduction in Clinical Global Impression-Improvement Scale (CGI)) within about 45, about 21, about 15, about 8, or about 3 days or less. In some embodiments, the therapeutic effect is a CGI score of 2 or less.

In some embodiments, the method provides therapeutic effect (e.g., as measured by reduction in Hamilton Anxiety Score (HAM-A)) within about 45, about 21, about 15, about 8, or about 3 days. HAM-A is scored where <17 indicates mild severity, 18-24 mild to moderate severity and 25-30 moderate to severe. In some embodiments, the therapeutic effect is a decrease from baseline in HAM-A score at the end of a treatment period (e.g., about 45, about 21, about 15, about 8, or about 3 days after beginning administration or episodic dosing). In some embodiments, the decrease from baseline in HAM-A score is from severe (e.g., HAM-A score of 25 or greater) to symptom-free (e.g., HAM-A score of 17 or lower). In some embodiments, the decrease from baseline in HAM-A score is from severe (e.g., HAM-A score of 25 or greater) to mild (e.g., HAM-A score of 24 or lower).

In some embodiments, the instruction set describes a method comprising an episodic dosing regimen, wherein the episodic dosing regimen occurs for about 2 weeks to about 6 weeks. In some embodiments, the instruction set describes a method comprising an episodic dosing regimen, wherein the episodic dosing regimen occurs for about 2 weeks to about 4 weeks. In some embodiments, the instruction set describes a method comprising an episodic dosing regimen, wherein the episodic dosing regimen occurs for about 2 weeks, or about 14 days. In some embodiments, the instruction set describes a method comprising an episodic dosing regimen, wherein the episodic dosing regimen occurs for 2 weeks.

In one embodiment, the instruction set is a printed instruction set.

In further embodiments, the instruction set describes a method comprising an episodic dosing regimen, wherein the method comprises administering Compound 1 to a subject concurrent with an episode of a disorder being treated. In some aspects, the instruction set describes a method comprising an episodic dosing regimen, wherein the method comprises administering Compound 1 to a subject concurrent with an episode of a disorder being treated, e.g., an episode of depression. In some aspects, the instruction set describes a method comprising an episodic dosing regimen, wherein the method comprises administering Compound 1 to a subject concurrent with an episode of a disorder being treated, e.g., an episode of depression. In some aspects, the instruction set describes a method comprising an episodic dosing regimen, wherein the method comprises administering Compound 1 to a subject concurrent with an episode of a disorder being treated, e.g., an episode of major depressive disorder, bipolar depression, anxiety, or postpartum depression. In some embodiments, the major depressive disorder is moderate major depressive disorder. In some embodiments, the major depressive disorder is severe major depressive disorder.

In an aspect, provided herein is a method of treating depression in a subject in need thereof, the method comprising the steps of:
(i) administering once daily to the subject a therapeutically effective amount of a compound having the formula:

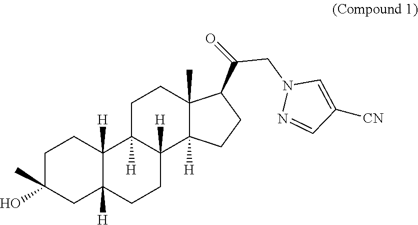

for about two weeks; and
(ii) re-administering once daily to the subject a therapeutically effective amount, e.g., about 30 mg, of Compound 1 for about two weeks in response to a recurrence of depression symptoms, provided there is at least a six week interval between administration of Compound 1 to the subject and re-administration of Compound 1 to the subject.

It is understood that the six week interval, as described above, is the duration between the last dose of administration of Compound 1 to the subject and first dose of re-administration of Compound 1 to the subject.

In some embodiments, Compound 1 is administered to the subject for 2 weeks. In some embodiments, Compound 1 is re-administered to the subject for 2 weeks. In some embodiments, the interval between administration of Compound 1 to the subject and re-administration of Compound 1 to the subject is 6 weeks. In some embodiments, the interval between administration of Compound 1 to the subject and re-administration of Compound 1 to the subject is 7 weeks. In some embodiments, the interval between administration of Compound 1 to the subject and re-administration of Compound 1 to the subject is 8 weeks.

In some embodiments, the depression is major depressive disorder (MDD). In some embodiments, the MDD is moderate major depressive disorder. In some embodiments, the MDD is severe major depressive disorder. In some embodiments, the depression is bipolar depression. In some embodiments, the depression is post-partum depression. In some embodiments, the subject has been diagnosed with depression. In some embodiments, the depression is major depressive disorder or bipolar depression. In some embodiments, the subject is a female diagnosed with severe postpartum depression. In some embodiments, the subject has been experiencing a major depressive episode over about a 1-year period. In some embodiments, the subject is between about 18 and about 75 years of age. In some embodiments, the subject is between about 18 and about 65 years of age.

In some embodiments, the subject is administered about 10 mg of Compound 1. In some embodiments, the subject is administered about 20 mg of Compound 1. In some embodiments, the subject is administered about 30 mg of Compound 1. In some embodiments, the subject is administered about 40 mg of Compound 1. In some embodiments, the subject is administered about 10 mg of Compound 1 once a day. In some embodiments, the subject is administered about 20 mg of Compound 1 once a day. In some embodiments, the subject is administered about 30 mg of Compound 1 once a day. In some embodiments, the subject is administered about 40 mg of Compound 1 once a day. In some embodiments, the subject is administered about 45 mg of Compound 1 once a day. In some embodiments, the amount of Compound 1 administered to the subject is reduced in the occurrence of a severe adverse effect. In some embodiments, Compound 1 is administered in the evening. In some embodiments, Compound 1 is administered with food. In some embodiments, Compound 1 is in a capsule. In some embodiments, the method further comprises administration of a second therapeutic agent.

In an aspect, provided herein is a method of treating major depressive disorder in a subject in need thereof, the method comprising the steps of:
(i) a first administration of a therapeutically effective amount of Compound 1, once daily, to the subject for 14 days:

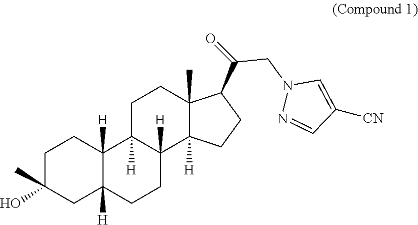

and
(ii) a second administration of a therapeutically effective amount of Compound 1 to the subject, once daily, in response to a recurrence of symptoms of major depressive disorder, provided there is at least a six week interval between the last dose of the first administration of Compound 1 to the subject and first dose of the second administration of Compound 1 to the subject.

In an aspect, provided herein is a method of treating postpartum depression in a subject in need thereof, the method comprising the steps of:
(i) a first administration of a therapeutically effective amount of Compound 1, once daily, to the subject for 14 days:

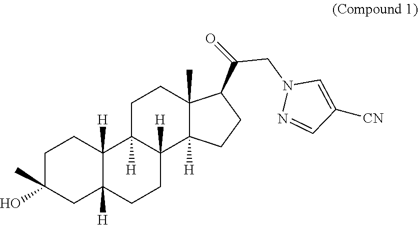

and
(ii) a second administration of a therapeutically effective amount of Compound 1 to the subject, once daily, in response to a recurrence of symptoms of major depressive disorder, provided there is at least a six week interval between the last dose of the first administration of Compound 1 to the subject and first dose of the second administration of Compound 1 to the subject.

In an aspect, provided herein is a method of treating generalized anxiety disorder in a subject in need thereof, the method comprising the steps of:
(i) a first administration of a therapeutically effective amount of Compound 1, once daily, to the subject for 14 days:

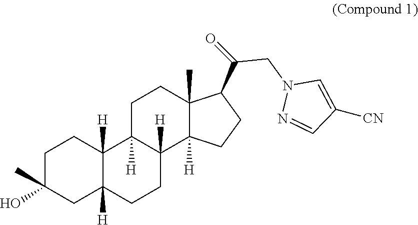

and
(ii) a second administration of a therapeutically effective amount of Compound 1 to the subject, once daily, in response to a recurrence of symptoms of major depressive disorder, provided there is at least a six week interval between the last dose of the first administration of Compound 1 to the subject and first dose of the second administration of Compound 1 to the subject.

In an aspect, provided herein is a method of treating bipolar depression in a subject in need thereof, the method comprising the steps of:
(i) a first administration of a therapeutically effective amount of Compound 1, once daily, to the subject for 14 days:

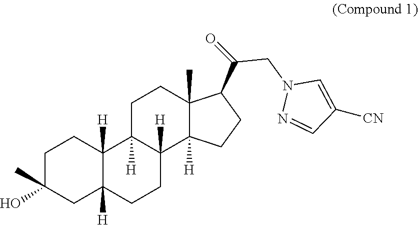

and
(ii) a second administration of a therapeutically effective amount of Compound 1 to the subject, once daily, in response to a recurrence of symptoms of major depressive disorder,
provided there is at least a six week interval between the last dose of the first administration of Compound 1 to the subject and first dose of the second administration of Compound 1 to the subject. In some aspects, provided herein are kits wherein a kit comprises an instruction set that describes a method for treating major depressive disorder, bipolar depression, anxiety, or postpartum depression by administering Compound 1, wherein the method comprises an episodic dosing regimen. In some embodiments, the major depressive disorder is moderate major depressive disorder. In some embodiments, the major depressive disorder is severe major depressive disorder. In some embodiments, about 10 mg, about 15 mg, about 20 mg, about 25 mg or about 30 mg of Compound 1 is administered to the subject. In some embodiments, Compound 1 is administered to the subject once a day for a plurality of weeks, e.g., about 2 weeks to about 6 weeks, e.g., about 2 weeks to about 4 weeks, e.g., about 2 weeks. In some embodiments, Compound 1 about 10 mg, about 15 mg, about 20 mg, about 25 mg or about 30 mg of Compound 1 is administered to the subject once a day for a plurality of weeks. In a preferred embodiment, the episodic dosing regimen occurs for about 2 weeks to about 6 weeks. In a more preferred embodiment, the episodic dosing regimen occurs for about 2 weeks to about 4 weeks. In an even more preferred embodiment, the episodic dosing regimen occurs for about 2 weeks, or about 14 days. In another embodiment, the episodic dosing regimen occurs for 2 weeks.

Also provided herein are methods of treating anxiety in a subject, the method comprising administering to the subject an effective amount of Compound 1 or a pharmaceutically acceptable salt thereof. Thus, in one aspect, provided herein is a method of treating anxiety in a subject, the method comprising administering to the subject a therapeutically effective amount of Compound 1 or a pharmaceutically acceptable salt thereof. In some embodiments, the method comprises administering to the subject a therapeutically effective amount of Compound 1. In some embodiments, the subject is between and including the ages of 18 and 64. In some embodiments, the compound is administered with food. In some embodiments, the therapeutically effective amount is 20 mg. In some embodiments, the therapeutically effective amount is 10 mg. In some embodiments, the therapeutically effective amount is 15 mg. In some embodiments, the therapeutically effective amount is 25 mg. In some embodiments, the therapeutically effective amount is about 30 mg. In some embodiments, the therapeutically effective amount is about 45 mg. In some embodiments, Compound 1 is administered in one or more capsules. In some embodiments, the therapeutically effective amount is administered across three capsules.

In some embodiments, the anxiety is generalized anxiety disorder. Generalized Anxiety Disorder (GAD) is characterized by persistent and excessive worry about a number of different things. People with GAD may anticipate disaster and may be overly concerned about money, health, family, work, or other issues. Individuals with GAD find it difficult to control their worry. They may worry more than seems warranted about actual events or may expect the worst even when there is no apparent reason for concern.

In other embodiments, the anxiety is obsessive-compulsive disorder (OCD); panic disorder, post-traumatic stress disorder (PTSD), or social anxiety disorder. Obsessive-Compulsive Disorder, OCD, is an anxiety disorder and is characterized by recurrent, unwanted thoughts (obsessions) and/or repetitive behaviors (compulsions). Repetitive behaviors such as hand washing, counting, checking, or cleaning are often performed with the hope of preventing obsessive thoughts or making them go away. Performing these so-called “rituals,” however, provides only temporary relief, and not performing them markedly increases anxiety. Panic disorder is an anxiety disorder and is characterized by unexpected and repeated episodes of intense fear accompanied by physical symptoms that may include chest pain, heart palpitations, shortness of breath, dizziness, or abdominal distress. Post-Traumatic Stress Disorder, PTSD, is an anxiety disorder that can develop after exposure to a terrifying event or ordeal in which grave physical harm occurred or was threatened. Traumatic events that may trigger PTSD include violent personal assaults, natural or human-caused disasters, accidents, or military combat. Social Phobia, or Social Anxiety Disorder, is an anxiety disorder characterized by overwhelming anxiety and excessive self-consciousness in everyday social situations. Social phobia can be limited to only one type of situation—such as a fear of speaking in formal or informal situations, or eating or drinking in front of others—or, in its most severe form, may be so broad that a person experiences symptoms almost anytime they are around other people.

Provided herein are methods for treating major depressive disorder, bipolar depression, anxiety, or postpartum depression with an episodic dosing regimen, comprising dosing of Compound 1 to a subject in need thereof. In some embodiments, the method improves cognitive function in the subject after completing the episodic dosing regimen. In some aspects, the method improves cognitive function in the subject after completing the episodic dosing regimen, wherein the episodic dosing regimen had a duration of about 2 to about 8 weeks. In further aspects, the method improves cognitive function in the subject after completing the episodic dosing regimen, wherein the episodic dosing regimen had a duration of about 2 to about 6 weeks. In other embodiments, the method improves cognitive function in the subject after completing the episodic dosing regimen, wherein the episodic dosing regimen had a duration of about 2 to about 4 weeks. In further embodiments, the method improves cognitive function in the subject after completing the episodic dosing regimen, wherein the episodic dosing regimen had a duration of about 2 weeks or 14 days. In other aspects, the method improves cognitive function in the subject after completing the episodic dosing regimen, wherein the episodic dosing regimen had a duration of 2 weeks.

Provided herein are methods for treating major depressive disorder, bipolar depression, anxiety, or postpartum depression with an episodic dosing regimen, comprising dosing of Compound 1 to a subject in need thereof. In some embodiments, the method provides no change in cognitive function in the subject after completing the episodic dosing regimen. In some embodiments, the method provides no cognitive impairment, or no change in cognitive function.

In some other aspects, described herein are kits comprising a plurality of individual dosage units of a pharmaceutical composition comprising Compound 1 and an instruction set, as described herein. In some embodiments, an instruction set describes a method for administering said pharmaceutical composition to a patient, wherein said method comprises an episodic dosing regimen. In another embodiment, the present invention provides a kit comprising:
1. A plurality of individual dosage units of a pharmaceutical composition comprising Compound 1; and
2. an instruction set for administering said dosage units to a patient in need thereof using an episodic dosing regimen.

In some embodiments, the instruction set is printed on a suitable material, such as paper In some embodiments, the dosage unit is a capsule. In some embodiments, the unit dosage, or dosage unit includes prefilled, premeasured ampules or syringes of a liquid compositions, or pills, tablets, capsules or the like in the case of solid compositions. In some embodiments, the dosage unit is a capsule of size 1. In other embodiments, the capsules are of size, 000, 00, 0, 1, 2, 3, or 4, as understood in the art.

### EXAMPLES

### Example 1: Compound 1 and Postpartum Depression

Compound 1 was investigated for use in subjects with depression. Female subjects (18-65 years old) diagnosed with severe postpartum depression (PDD) with a HAM-D total score of greater than or equal to 26 at screening and Day 1 were used in the investigation. The subjects were dosed once per day with capsules containing 30 mg of Compound 1. The dose could be adjusted to capsules containing 20 mg of Compound 1 if the 30 mg dose was not tolerated. Subjects not dosed with Compound 1 were dosed with a placebo. In total, 78 subjects were treated with Compound 1 at 30 mg and 73 subjects were treated with placebo.

### Statistics

Assuming a 2-sided test at an alpha level of 0.05, a sample size of approximately 65 evaluable subjects per treatment group provided 90% power to detect a placebo-adjusted treatment difference of approximately 4 points in the primary endpoint, change from baseline in HAM-D total score at Day 15 assuming standard deviation (SD) of 7 points. The change from baseline in the HAM-D total score was analyzed using a mixed effects model for repeated measures (MMRM). The model included the change from baseline at each visit as the dependent variable. The main comparison was (difference in least mean square [LSMEAN]) between Compound 1 Capsules and placebo at the 15-day timepoint.

For Model-based point estimates (i.e., LSMEAN, 95% confidence intervals, and p-values), an unstructured covariance structure was used to model the within-subject errors. If there was a convergence issue with the unstructured covariance model, Toeplitz, compound symmetry or Autoregressive (1) (AR[1]) covariance structure was used, following this sequence until convergence was achieved. If the model still does not converge with AR(1) structure, no results were reported. When the covariance structure was not UN, the sandwich estimator for the variance-covariance matrix was derived, using the EMPIRICAL option in the PROC MIXED statement in SAS.

Similarly, an MMRM was used for the analysis of the following variables: changes from baseline in MADRS total score and HAM-A total score, and select individual item and subscale scores. For each model, the comparison of interest was between Compound 1 Capsules and matching placebo at the 15-day time point. Model-based point estimates (i.e., LS means), 95% confidence intervals, and p-values were reported.

### Results

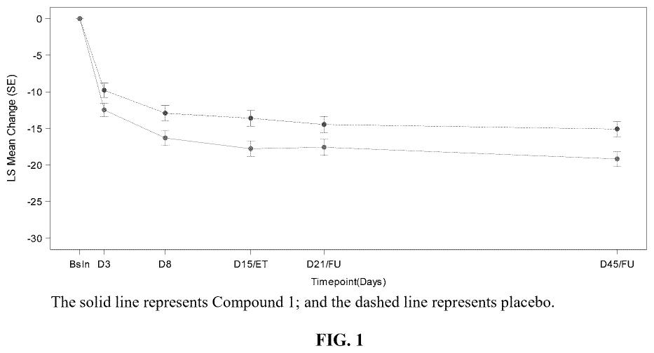

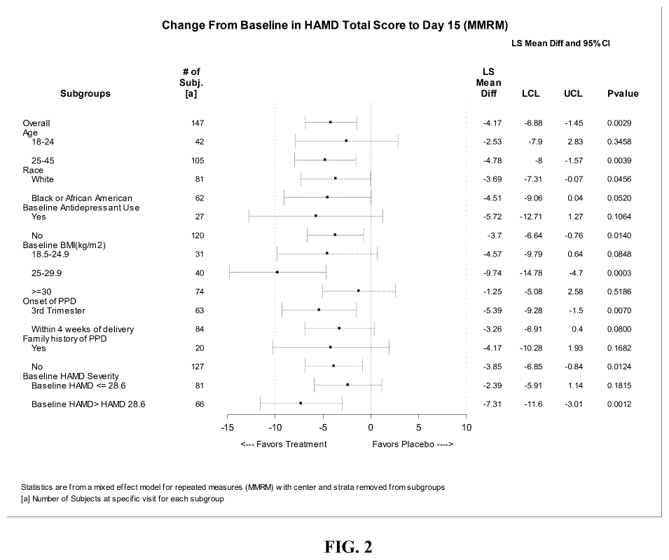

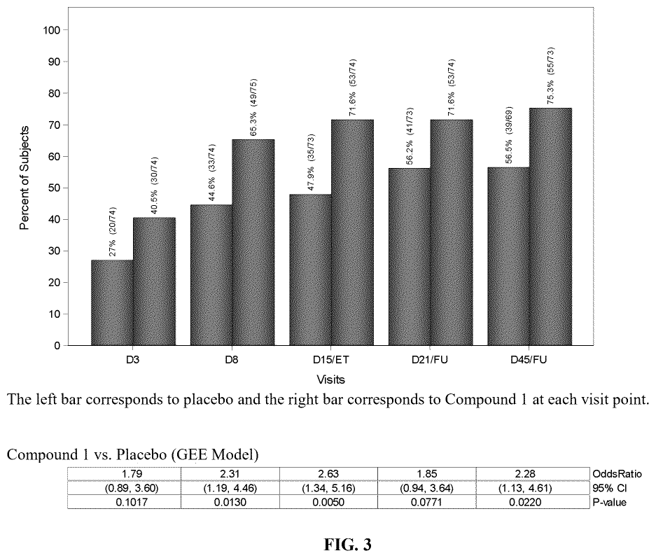

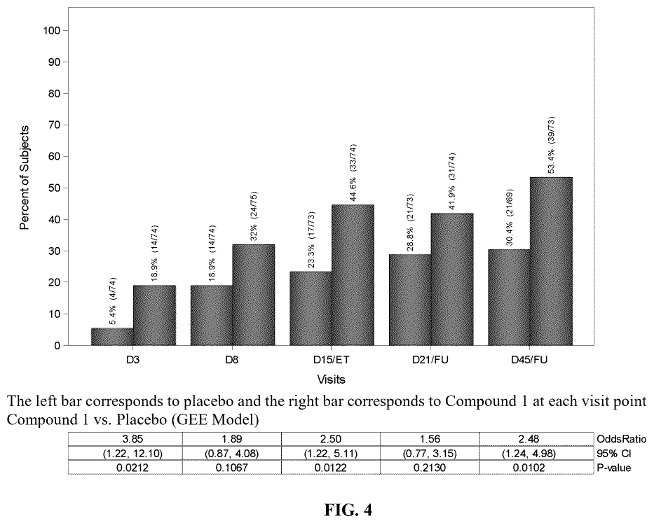

The results demonstrate that the primary endpoint was met. The mean decrease from baseline in HAM-D total score was −18.0 (8.36) for Compound 1 (30 mg) treated subjects from mean baseline of 28.4 (2.09) and −13.6 (8.31) for placebo treated subjects from mean baseline of 28.8 (2.32) at Day 15. The model-based between treatment group difference at Day 15 and corresponding 95% confidence internal (CI) was −4.2 (−6.9, −1.5) in favor of Compound 1, p-value-0.0029. FIG. 1 depicts the LS mean change from baseline in Hamilton Rating Scale for Depression (HAM-D), total score over time by treatment group. FIG. 2 depicts a forest plot of the subgroup analysis for primary endpoint at day 15. FIG. 3 depicts a bar chart of Hamilton Rating Scale for Depression (HAM-D) remission by time point and treatment group. FIG. 4 depicts a bar chart of Hamilton Rating Scale for Depression (HAM-D) remission by time point and treatment group.

Rates of HAM-D Response and Remission were Significantly Higher in Subjects Treated with Compound 1 vs. Those with Placebo:
Response: 53/74 (71.6%) for Compound 1 treated subjects (30 mg) vs. 35/73 (47.9%) for placebo treated subjects. The model-based odds ratio and corresponding (95% CI) was 2.63 (1.34, 5.16), p-value=0.0050.
Remission: 33/74 (44.6%) for Compound 1 treated subjects (30 mg) vs. 17/73 (23.3%) for placebo treated subjects. The model-based odds ratio and corresponding (95% CI) was 2.50 (1.22, 5.11), p-value=0.0122.
   Change in MADRS Total Score at Day 15 and all Other Timepoints from Baseline:

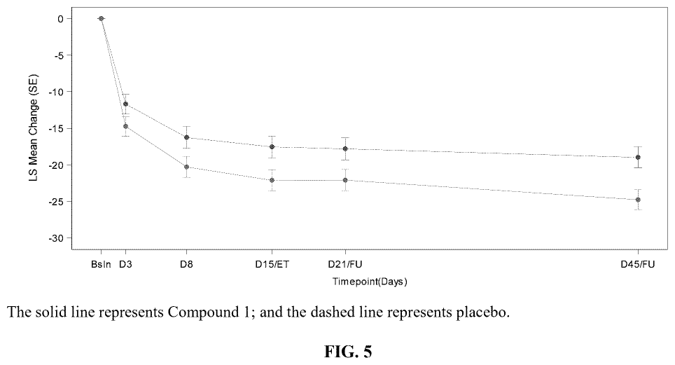

The mean decrease from baseline in MADRS total score was −22.0 (11.64) for Compound 1 treated subjects (30 mg) from baseline of 34.9 (4.41) and −17.7 (11.72) for placebo treated subjects from baseline of 36.3 (4.68) at Day 15. The model-based between treatment group difference and corresponding 95% confidence interval (CI) was −4.6 (−8.3, −0.8) favoring Compound 1, p-value=0.0182. The results from the study are shown in FIG. 5.

Change in HAM-A Total Score at Day 15 and all Other Timepoints from Baseline:

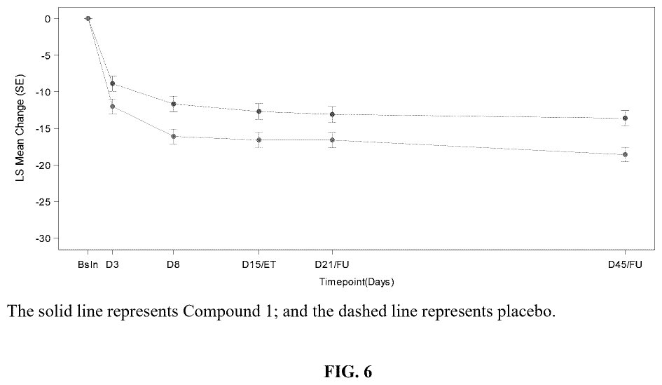

The mean decrease from baseline in HAMA total score was −16.5 (9.51) for Compound 1 (30 mg) treated subjects from mean baseline of 26.1 (5.88), and −12.9 (8.57) for placebo treated subjects from baseline of 27.2 (5.45) at Day 15. The model-based between treatment group difference and corresponding 95% confidence internal (CI) was −3.90 (−6.7, −1.1) favoring Compound 1, p-value-0.0063. The results from the study are shown in FIG. 6.

### CGI-I Response at Day 15:

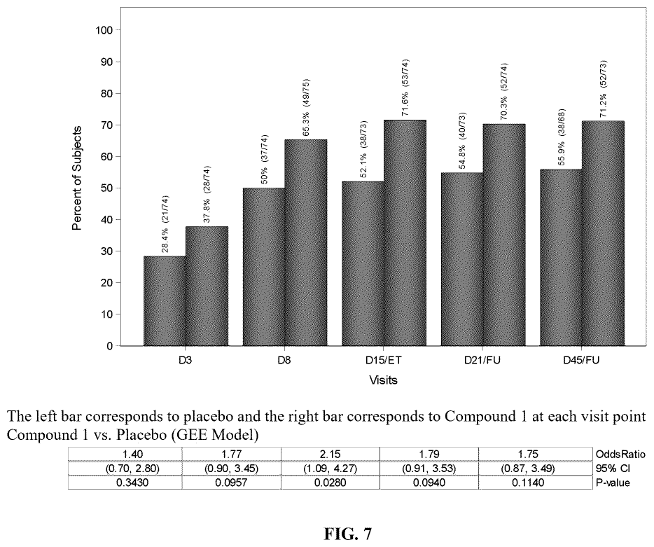

53/74 (71.6%) for Compound 1 (30 mg) treated subjects vs. 38/73 (52.1%) for placebo treated subjects. The model-based odds ratio and corresponding (95% CI) was 2.15 (1.09, 4.27) favoring Compound 1, p-value=0.0280. The results from the study described herein are shown in FIG. 7.

The study demonstrated that Compound 1 administered at 30 mg once a day for 15 days (an exemplary episodic dosing regimen) was effective at treating postpartum depression when compared to placebo.

### Example 2: A Phase 3, Open-Label, 1-Year Study of the Safety, Tolerability, and Need for Re-Treatment with Compound 1 in Adult Subjects with Major Depressive Disorder (MDD)

### List of Abbreviations

ADT Antidepressant therapy
AE adverse event
CGI-I Clinical Global Impression-Improvement
CGI-S Clinical Global Impression-Severity
CS clinically significant
C-SSRS Columbia Suicide Severity Rating Scale
CYP cytochrome P450
DSM-5 Diagnostic and Statistical Manual of Mental Disorders, Fifth Edition
ECG electrocardiogram
eCRF electronic case report form
EOT end of treatment
ET early termination
FSH follicle stimulating hormone
GABA γ-aminobutyric acid
HAM-D Hamilton Rating Scale for Depression
HCV hepatitis C virus
HIV human immunodeficiency virus
ICF informed consent form
IRB institutional review board
IRT interactive response technology
ISI Insomnia Severity Index
MADRS Montgomery-Åsberg Depression Rating Scale
MDD major depressive disorder
MDE major depressive episode
NCS not clinically significant
PHQ-9 9-item Patient Health Questionnaire
PK pharmacokinetic(s)
PSQ patient status questionnaire
QTcF QT corrected according to Fridericia's formula
SAE serious adverse event
SAP statistical analysis plan
SCID-5-CT Structured Clinical Interview for Diagnostic and DSM-5
SD standard deviation
SDS Sheehan Disability Scale
SUSAR suspected, unexpected, serious adverse reactions
TEAE treatment-emergent adverse event
WHO World Health Organization

### Overall Study Design

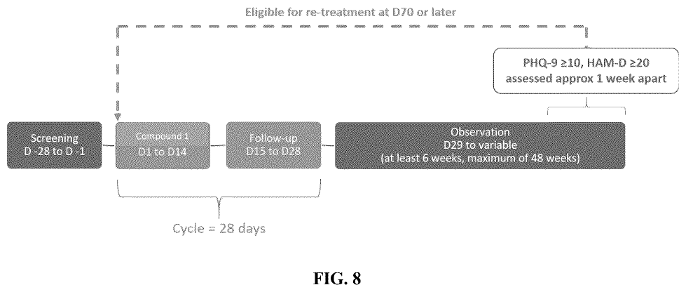

Compound 1 was investigated in an open-label, long-term, longitudinal study in adult subjects with MDD currently experiencing a major depressive episode (MDE). See FIG. 8 for a schematic of the study design.

The diagnosis of MDD was made according to Structured Clinical Interview for DSM-5 Clinical Trial Version (SCID-5-CT), performed by a qualified healthcare professional. Subjects were evaluated in a preliminary screening procedure at the Screening Visit to determine eligibility, including completion of the MADRS and CGI-S.

The primary objective of the study was to determine the safety and tolerability of initial treatment and re-treatment(s) with Compound 1 in adults with MDD currently experiencing a major depressive episode (MDE) over a 1-year period.

Secondary objectives of the study were to assess the need for re-treatment with Compound 1 following initial treatment in adults with MDD currently experiencing an MDE over a 1-year period and to assess the response of initial treatment and re-treatment(s) with Compound 1 following an initial 2-week treatment period (an exemplary episodic dosing regimen) in adults with MDD currently experiencing an MDE over a 1-year period.

Exploratory objectives of the study were to develop a digital phenotype of adults with MDD currently experiencing an MDE and assess potential correlations with clinical endpoints; assess the effect of Compound 1 on sleep; and assess patient-reported outcome measures as they relate to impact of depression on subjects' lives, severity of depression, functionality, subject perspective of symptoms, and subject satisfaction with Compound 1.

The primary endpoint of the study was the safety and tolerability of the initial treatment with Compound 1 and re-treatment with Compound 1, as assessed by measures including the incidence and severity of AEs/SAEs; changes from baseline in clinical laboratory measures, vital signs, and electrocardiograms (ECGs); and suicidal ideation and behavior using the Columbia Suicide Severity Rating Scale (C-SSRS).

Secondary endpoints of this study were: the need for re-treatment with Compound 1 as assessed by: time to first re-treatment (Kaplan-Meier curves); number of subjects achieving the requirements for re-treatment; and number of re-treatment cycles for each subject. The response of initial treatment and/or re-treatment as assessed by change from baseline in the 17-item HAM-D total score at the end of each 14-day treatment (initial and/or re-treatment) period; HAM-D response at the end of each 14-day treatment (initial and/or re-treatment); period, defined as a ≥50% reduction in HAM-D score from baseline; HAM-D remission at the end of each 14-day treatment (initial and/or re-treatment) period, defined as HAM-D total score ≤7; CGI-I response, defined as “much improved” or “very much improved”, at the end of each 14-day treatment (initial and/or re-treatment) period; and change from baseline in Clinical Global Impression-Severity (CGI-S) score at the end of each 14-day treatment (initial and/or re-treatment) period (also referred to as exemplary episodic dosing regimen(s)).

Exploratory endpoints of this study were: digital phenotype as developed by passive collection of basic behavior data, such as such as GPS, text/phone use, motor activity/sleep patterns in subjects who provide consent to use a mobile phone-supported software application; effect of Compound 1 on sleep as assessed by the Insomnia Severity Index (ISI); time to first new ADT use (Kaplan-Meier curves) and number of new ADTs used; patient-reported depressive symptoms as assessed by the 9-item Patient Health Questionnaire (PHQ-9); patient-reported functionality as assessed by the Sheehan Disability Scale (SDS); and patient-reported impact of depression and patient perspective of symptoms and satisfaction as assessed by a patient status questionnaire (PSQ).

The duration of subject participation was approximately 56 weeks: Screening Period (28 days), Initial Treatment Period (14 days, or exemplary episodic dosing regimen), Follow-up Period (14 days), and Observational Period (48 weeks). Additional 14-day re-treatment periods (or episodic dosing regimen) with Compound 1 may have occurred during the 48-week Observational Period.

All subjects received a daily oral dose of Compound 1 from Day 1 through Day 14 of the first treatment cycle. According to the re-emergence or recurrence, or reappearance of depressive symptoms, Compound 1 was administered in subsequent 14-day treatment periods (re-administration or further episodic dosing regimen).

Subjects Achieving Response with Compound 1 Followed for 48 Weeks

Beginning on Day 1, qualified subjects self-administered 30 mg of Compound 1 orally once daily in the evening for 14 days. A follow-up visit was conducted 14 days (±1 day) after the completion of the 14-day treatment period.

If a subject did not exhibit a response to Compound 1 by Day 15 of the initial treatment, defined as a ≥50% reduction in HAM-D score from baseline, the subject was terminated from the study upon completion of the 14-day follow-up period.

After the initial treatment period, subjects were followed naturalistically for 48 weeks. Subjects returned to the site every 8 weeks (beginning after the first follow-up period) during the 48-week observational period for clinical assessments.

### Compound 1 Treatment Cycles

Each 14-day treatment period of Compound 1 and corresponding 14-day follow-up period was considered a cycle (Day 28). The initial treatment was Cycle 1 and re-treatments were numbered sequentially. Each cycle began with Day 1 (e.g., the first day of the first re-treatment period was Day 1 of Cycle 2). A maximum of 5 treatment cycles was permitted; a new re-treatment cycle did not start after Week 48. Subjects starting a new Compound 1 treatment cycle between Weeks 45 and 48 were followed through the end of the treatment cycle (Day 28, end of Follow-up period of treatment cycle).

The need for re-treatment was assessed every 14 days via remote assessments during the 48-week observational period based on the results of the subject-reported PHQ-9; if the PHQ-9 score was ≥10, the subject returned to the site to be assessed by the clinician-administered HAM-D. New Compound 1 cycles were initiated for subjects with a HAM-D score >20 assessed approximately 1 week from the PHQ-9 score ≥10.

A minimum period or interval of 8 weeks (56 days) was required between Compound 1 treatment cycles. This was based on the period of 8 weeks establishing ‘full remission’ of a depressive episode (American Psychiatric Association 2013) and is aligned with the treatment period which would be required for any available antidepressant (ADT) to exhibit maximal efficacy.

As this was the first study in which longitudinal re-treatment with Compound 1 would be examined, and based on known withdrawal symptoms with other GABAergic drugs and non-clinical findings in a 9-month study of Compound 1 in dogs, the potential for withdrawal-related events, including seizure, was monitored.

### Study Drug Packaging and Labeling

Compound 1 was provided to the clinic pharmacist and/or designated site staff responsible for dispensing the study drug in appropriately labeled, subject-specific kits containing sealed unit doses. Each unit dose consists of 1 capsule.

### Study Drug Administration

Compound 1 was administered orally once daily in the evening with food. Practical options included taking Compound 1 within 1 hour of dinner or taking Compound 1 later in the evening with solid food. If a subject misses a dose, the subject skipped that dose (i.e., they should not take the dose in the morning) and took the next scheduled dose the next evening. As this was the first study in which longitudinal re-treatment with Compound 1 was examined, and based on known withdrawal symptoms with other GABAergic drugs and non-clinical findings in a 9-month study of Compound 1 in dogs (Investigator's Brochure), the potential for withdrawal-related events, including seizure, was monitored, which included study drug discontinuation or dose reduction.

If a subject exhibited suicidality at any time, they returned to the site as soon as possible for assessment by the Investigator.

The assessments for the Screening Period and Treatment and Follow-up Periods are summarized in Table 1; the assessments for the observational period and any unscheduled visits are summarized in Table 2.

TABLE 1

| | | Cycle^{c} | | | |
|---|---|---|---|---|---|
| | Screening | Open-label Treatment Period | | | |
| | Period^{a, b} | (Initial and Re-treatments) | | | Follow-up |
| | Days | | | | |
| | | | | D 15 | D 28 |
| | D −28 | | D 8 | (±1 d)/ | (±1 d) |
| Study Procedure | to D −1 | D 1 | (+1 d) | EOT^{d} | and/or ET |
| Informed Consent | X | | | | |
| Duplicate Subject Check^{e} | X | | | | |
| Inclusion/Exclusion | X | X | | | |
| Demographics | X | | | | |
| Medical/Family History | X | | | | |
| SCID-5 | X | | | | |
| ICD-10 | X | | | | |
| MGH ATRQ | X | | | | |
| Serum FSH test^{f} | X | | | | |
| Physical Examination^{g} | X | X | | | |
| Body Weight/Height | X | | | X (wt only) | |
| Clinical Laboratory Assessments^{h} | X | X | X | X | |
| Drug & Alcohol Screenⁱ | X | X | X | X | |
| Pregnancy Test^{j} | X | X | | X^{k} | |
| Hepatitis & HIV Screen | X | | | | |
| Exploratory Blood Sample^{l} | O | | O | O | |
| Exploratory Genetic Sample^{m} | O | | | | |
| Vital Signsⁿ | X | X | X | X | X |
| 12-Lead ECG^{o} | X | X | | X | |
| C-SSRS^{p} | X | X | X | X | X |
| MADRS | X | X | | | |
| HAM-D^{q, r} | | X | X | X | X^{s} |
| CGI-S | X | X | X | X | X |
| CGI-I | | | X | X | X |
| PHQ-9 | | X | X | X | X |
| SDS | | X | | X | X |
| PSQ | | X | | X | |
| ISI | | X | X | X | X |
| Study Drug Dispensation | | X | X | | |
| Study Drug Administration | | | X (Day 1 through | | |
| | | | Day 14) | | |
| Study Drug Accountability/Return | | | X | X | |
| Digital Phenotyping (Mobile device | | | O | | |
| application)^{b, t} | | | | | |
| Adverse Events/SAEsu | | | X | | |
| Prior/Concomitant Medicationsv | | | X | | |

CGI-I = Clinical Global Impression - Improvement; CGI-S—Clinical Global Impression - Severity; C-SSRS = Columbia Suicide Severity Rating Scale; D = day; ET = early termination; ECG = electrocardiogram; EOT = end of treatment; FSH = follicle stimulating hormone; HAM-D = Hamilton Rating Scale for Depression, 17-item; HIV = human immunodeficiency virus; ICD-10 = International Statistical Classification of Diseases and Related Health Problems version 10; ISI = Insomnia Severity Index; MADRS = Montgomery-Åsberg Depression Rating Scale; MGH ATRQ = Massachusetts General Hospital Antidepressant Treatment Response Questionnaire; O = Optional; SCID-5 = Structured Clinical Interview for Diagnostic and Statistical Manual of Mental Disorders, Fifth Edition; PHQ-9 = 9-item Patient Health Questionnaire; PSQ = patient status questionnaire; SAE = serious adverse event; SDS = Sheehan Disability Scale; wt = weight
^{a}Screening procedures are to be conducted before initial (Cycle 1) treatment period only.
^{b}A minimum of 14 days of Screening is required for subjects that consent to use the mobile phone-supported software application for digital phenotyping.
^{c}Each cycle is 28 days (±1 day) and is comprised of a 14-day treatment period and a 14-day follow-up period. The initial treatment is considered Cycle 1 and re-treatments will be numbered sequentially. Each re-treatment cycle will begin with Day 1 (eg, the first day of the first re-treatment will be Day 1 of Cycle 2).
^{d}Subjects who discontinue treatment early should return to the site for an end of treatment (EOT) visit as soon as possible, preferably the day after treatment is discontinued. The follow-up visits should occur 14 days after the last dose of treatment. If at any time after the EOT visit, a subject decides to terminate the study, the subject should return for an early termination (ET) visit. The EOT and ET visits can be on the same day if a subject discontinues study drug and terminates the study on the same day during a clinic visit; in this case, all events scheduled for the EOT visit will be conducted.
^{e}Subjects will be asked to authorize that their unique subject identifiers be entered into a registry (www.subjectregistry.com) with the intent of identifying subjects who may meet exclusion criteria for participation in another clinical study.
^{f}A serum FSH test will be conducted at Screening for female subjects that are not surgically sterile to confirm whether a female subject with ≥12 months of spontaneous amenorrhea meets the protocol-defined criteria for being post-menopausal.
^{g}A full physical examination will be conducted at Screening and abbreviated physical examinations will be conducted thereafter. A full physical examination includes assessment of body systems (eg, head, eye, ear, nose, and throat; heart; lungs; abdomen; and extremities).
^{h}Safety laboratory tests will include hematology, serum chemistry, coagulation, and urinalysis.
ⁱUrine toxicology for selected drugs of abuse (as per the laboratory manual) and breath test for alcohol.
^{j}Serum pregnancy test at screening and urine pregnancy test thereafter.
^{k}Female subjects who prematurely discontinue will have a pregnancy test performed at the EOT visit.
^{l}An optional blood sample for hormone and exploratory biochemistry testing, where consent is given.
^{m}An optional genetic sample for biomarker testing, where consent is given.
ⁿVital signs include oral temperature (° C.), respiratory rate, heart rate, and blood pressure (supine and standing). Heart rate and blood pressure to be collected in supine position at all scheduled time points after the subject has been resting for 5 minutes and then in the standing position. Vital signs may be repeated at the discretion of the Investigator as clinically indicated.
^{o}Triplicate ECGs will be collected.
^{p}The “Baseline/Screening” C-SSRS form will be completed at screening. The “Since Last Visit” C-SSRS form will be completed at any time of day at all subsequent time points.
^{q}The HAM-D is to be completed as early during the visit as possible.
^{r}The assessment timeframe for the HAM-D scale will refer to the past 7 days (1 week).
^{s}Subjects that do not exhibit a response to Compound 1 by Day 15 of the initial treatment, defined as a ≥50% reduction in HAM-D score from baseline, will be terminated from the study upon completion of the follow-up visit.
^{t}Subjects who provide consent will use a mobile-phone supported software application beginning at the Screening visit through the duration of the study.
^{u}Adverse events will be collected starting at the time of informed consent and throughout the duration of the subject's participation in the study.
^{v}Prior medications will be collected at Screening and concomitant medications will be collected at each subsequent visit.

TABLE 2

| | Observational Period^{a} | | |
|---|---|---|---|
| | (Day 29 through Week 52) | | |
| | Remote | Visit | Unscheduled |
| | Assessment | Q8W/ET | Visit |
| Study Procedure | Q2W (±1 d) | (±3 d) | (as needed)^{b} |
| PHQ-9^{c} | X^{d} | X | X |
| Physical Examination | | X | |
| Body Weight | | X | |
| Clinical Laboratory Assessments | | X | |
| Drug & Alcohol Screen | | X | |
| Pregnancy Test | | X | |
| Vital Signs | | X | |
| 12-Lead ECG | | X | |
| C-SSRS | | X | |
| HAM-D^{e} | | X | X |
| CGI-S | | X | |
| CGI-I | | X | |
| SDS | | X | X |
| ISI | | X | X |
| Concomitant | | X | X |
| Medications^{f} | | | |
| Digital Phenotyping | | O | |
| (Mobile device | | | |
| application)^{g} | | | |
| Adverse Events/SAEs^{h} | | X | |

ECG = electrocardiogram; ET = early termination; HAM-D = Hamilton Rating Scale for Depression, 17-item; ISI = Insomnia Severity Index; O = optional; PHQ-9 = 9-item Patient Health Questionnaire; PSQ = patient status questionnaire; Q2W = once every 2 weeks; Q8W = once every 8 weeks; SDS = Sheehan Disability Scale; SAE = serious adverse event
^{a}The schedule of the assessments in the Observational Period should be based on the last day of the preceding treatment cycle (eg, the first of the Q2W remote assessments will be on Day 42 (±1 day) and the first of the Q8W visits will be on Day 84 (±3 days)).
^{b}A subject will return to the site outside of the Q8W visit schedule if the PHQ-9 score is ≥10 and/or upon any suicidal thoughts or behaviors.
^{c}All PHQ-9 assessments will be performed via a mobile phone-supported software application.
^{d}The subject will take the PHQ-9 every 14 days; if the PHQ-9 score is ≥10, then the subject will return to the site to be assessed by the clinician-administered HAM-D in approximately one week. If the HAM-D score is <20. the subject will take the PHQ-9 on a weekly basis: the subject will return to the site to be assessed by the HAM-D each week that the PHQ-9 score remains ≥10; if the PHQ-9 score is <10, the subject will take the PHQ-9 every 2 weeks thereafter.
eIf the HAM-D score is ≥20 (assessed approximately one week from having a PHQ-9 score ≥10) and it has been at least 8 weeks since the last treatment day of the previous Compound 1 treatment cycle (ie, Day 70 or later), the subject will begin a 14-day re-treatment period with a 14-day follow-up visit (see Table 1). If the HAM-D score is ≥20 but it has been less than 8 weeks since the last treatment day of the previous Compound 1 treatment cycle (ie, Day 69 or earlier); the subject will take the PHQ-9 on a weekly basis until the 8-week period has lapsed, at which time the subject may begin a re-treatment period with Compound 1 (see Table 1), or until the PHQ-9 score is <10.
^{f}Concomitant medications will be collected at each in-clinic visit.
^{g}Subjects who provide consent for digital phenotyping will use a mobile phone-supported software application beginning at the Screening visit through the duration of the study.
^{h}Adverse events will be collected starting at the time of informed consent and throughout the duration of the subject's participation in the study.

### Dose Justification

The dose level in this study of 30 mg per day was the dose level that was efficacious and well-tolerated in a Phase 2 study in subjects with MDD. Dose adjustments to 20-mg of Compound 1 were permitted; 20-mg of Compound 1 was anticipated to be well tolerated as it was lower than the maximum tolerated dose level. Due to sedation/somnolence observed in previous clinical trials when administered in the morning, and improved tolerability when given in the evening, Compound 1 was administered in the evening in this study.

According to the DSM-5, a period of 8 weeks is required to establish ‘full remission’ of a depressive episode (American Psychiatric Association 2013). Further, available antidepressant therapies (ADT) often take up to 8 weeks to exhibit maximal efficacy. Thus, a minimum period of 8 weeks (56 days) was required between the end of a 14-day treatment period and the beginning of a new Compound 1 treatment cycle.

### Dose Adjustment Criteria

If at any time, 30 mg of Compound 1 was not tolerated, as assessed by the occurrence of a severe AE judged by the Investigator to be related to study drug, the dose was reduced to 20 mg as soon as possible and continued for the remainder of the treatment period. Dose adjustments related to moderate AEs were judged by the Investigator. If a dose adjustment from 30 mg to 20 mg was deemed necessary by the Investigator, the subject returned to the site for the adjusted dose to be dispensed. Any re-treatment period began with the 30-mg dose, regardless of whether a subject required a dose adjustment in a previous treatment period. Subjects who did not tolerate the 20-mg dose at any time were discontinued from study drug and the subject was terminated from the study upon completion of the subsequent 14-day follow-up period.

### Subject Inclusion Criteria

Qualified subjects met all of the following criteria:
1. Subject had signed an ICF prior to any study-specific procedures being performed.
2. Subject was a male or female between 18 and 75 years of age, inclusive.
3. Subject was in good physical health and has no clinically significant findings, as determined by the Investigator, on physical examination, 12-lead ECG, or clinical laboratory tests.
4. Subject agreed to adhere to the study requirements.
5. Subject has a diagnosis of MDD as diagnosed by SCID-5-CT, with symptoms that have been present for at least a 4-week period.
6. Subject has a MADRS total score of ≥28 at screening and Day 1 (prior to dosing).
7. Subjects taking antidepressants used to treat major depressive disorder must have been taking these medications at the same dose for at least 60 days prior to Day 1.
8. Female subject agreed to use one of the following methods of contraception during participation in the study and for 30 days following the last dose of study drug, unless they were postmenopausal (defined as no menses for 12 months without an alternative medical cause and confirmed by follicular stimulation hormone [FSH] >40 mIU/mL), surgically sterile (hysterectomy or bilateral oophorectomy), or does not engage in sexual relations which carry a risk of pregnancy: combined (estrogen and progestogen containing) oral, intravaginal, or transdermal hormonal contraception associated with inhibition of ovulation; oral, injectable, or implantable progestogen-only hormonal contraception associated with inhibition of ovulation; intrauterine device; Intrauterine hormone-releasing system; Bilateral tubal ligation/occlusion; Vasectomized partner; Sexual abstinence (no sexual intercourse).
9. Male subject agreed to use an acceptable method of effective contraception for the duration of study and for 5 days after receiving the last dose of the study drug, unless the subject does not engage in sexual relations which carry a risk of pregnancy. Acceptable methods of effective contraception for males includes sexual abstinence, vasectomy, or a condom with spermicide used together with highly effective female contraception methods if the female partner(s) is of child-bearing potential (see Inclusion Criteria #8 for acceptable contraception methods).
10. Male subject was willing to abstain from sperm donation for the duration of the study and for 5 days after receiving the last dose of the study drug.
11. Subject agreed to refrain from drugs of abuse and alcohol for the duration of the study.

### Subject Exclusion Criteria

Subjects who meet any of the following criteria were disqualified from participation in this study:
1. Subject had attempted suicide associated with the current episode of MDD.
2. Subject had a recent history or active clinically significant manifestations of metabolic, hepatic, renal, hematological, pulmonary, cardiovascular, gastrointestinal, musculoskeletal, dermatological, urogenital, neurological, or eyes, ears, nose, and throat disorders, or any other acute or chronic condition that, in the Investigator's opinion, would limit the subject's ability to complete or participate in this clinical study.
3. Subject had treatment-resistant depression, defined as persistent depressive symptoms despite treatment with adequate doses of antidepressants within the current major depressive episode (excluding antipsychotics) from two different classes for at least 4 weeks of treatment. Massachusetts General Hospital Antidepressant Treatment Response Questionnaire was used for this purpose.
4. Subject had had vagus nerve stimulation, electroconvulsive therapy, or has taken ketamine within the current major depressive episode.
5. Subject was taking benzodiazepines, barbiturates, or GABA_{A} modulators (e.g., eszopiclone, zopiclone, zaleplon, and zolpidem) at Day-28, or subjects have been using these agents daily or near-daily (>4 times per week) for more than one year.
6. Subject was taking non-GABA anti-insomnia medications (e.g., melatonin, Benadryl [anti-histamines], trazodone, low dose quetiapine, mirtazapine, etc.) and/or atypical antipsychotics (e.g., aripiprazole, quetiapine) at Day-14.
7. Subject had a known allergy to Compound 1, allopregnanolone, or related compounds.
8. Subject had a positive pregnancy test at screening or on Day 1 prior to the start of study drug administration for any treatment cycle.
9. Subject that was breastfeeding at Screening or on Day 1 (prior to administration of study drug) did not agree to temporarily cease giving breast milk to her child (ren) from just prior to receiving study drug on Day 1 until 7 days after the last dose of study drug in each treatment cycle.
10. Subject had detectable hepatitis B surface antigen, anti-hepatitis C virus (HCV) and positive HCV viral load, or human immunodeficiency virus (HIV) antibody at screening.
11. Subject had a clinically significant abnormal 12-lead ECG at the screening or baseline visits. NOTE: mean QT interval calculated using the Fridericia method (QTcF) of >450 msec in males or >470 msec in females were the basis for exclusion from the study.
12. Subject had active psychosis per Investigator assessment.
13. Subject had a medical history of seizures.
14. Subject had a medical history of bipolar disorder, schizophrenia, and/or schizoaffective disorder.
15. Subject had a history of mild, moderate, or severe substance use disorder (including benzodiazepines) diagnosed using DSM-5 criteria in the 12 months prior to screening.
16. Subject had been taking chronic or as-needed psychostimulants (e.g., methylphenidate, amphetamine) or opioids at Day −28.
17. Subject had had exposure to another investigational medication or device within 30 days prior to screening.
18. Subject had previously participated in a Compound 1 or a brexanolone clinical trial.
19. Use of any known strong inhibitors of cytochrome P450 (CYP)3A4 within 28 days or 5 half-lives (whichever was longer) or consumed grapefruit juice, grapefruit, or Seville oranges, or products containing these within 14 days prior to the first dose of study drug for any Compound 1 treatment cycle.
20. Use of the following strong CYP3A4 inducers within 28 days prior to the first dose of study drug for any Compound 1 treatment cycle: rifampin, carbamazepine, enzalutamide, mitotane, phenytoin, and St John's Wort.
21. Subject had a positive drug and/or alcohol screen at screening or on Day 1 prior to dosing of the initial treatment cycle.
22. Subject planned to undergo elective surgery during the initial treatment and follow-up period.
23. Subject had been diagnosed with and/or treated for any type of cancer (excluding basal cell carcinoma and in situ melanoma) within the past year prior to Screening.
24. Subject had a history of sleep apnea.
25. Subject had had gastric bypass surgery, has a gastric sleeve or lap band, or has had any related procedures that interfere with gastrointestinal transit.

### Subject Withdrawal Criteria

Subjects could withdraw from the study drug or terminate from the study at any time for any reason. The Investigator could withdraw the subject from the study drug or from the study for any of the following reasons: the subject was unwilling or unable to adhere to the protocol; the subject experiences an intolerable AE; other medical or safety reason, at the discretion of the Investigator and/or the Medical Monitor.

The Investigator notified the Sponsor and/or the Medical Monitor immediately when a subject withdrew from the study drug or terminated the study for any reason. The reason was recorded in the subject's electronic case report form (eCRF).

If a subject was persistently noncompliant, the Investigator discussed with the Sponsor the potential discontinuation of the subject. Any reasons for unwillingness or inability to adhere to the protocol was recorded in the subject's eCRF, including: missed visits, interruptions in the schedule of study drug administration, non-permitted medications.

Subjects who discontinued the study due to an AE, regardless of Investigator-determined causality, were followed until the event was resolved, considered stable, or the Investigator determined the event was no longer clinically significant.

Subjects who discontinued study drug early during a treatment period returned to the site for an end of treatment (EOT) visit as soon as possible, preferably the day after treatment was discontinued. The follow-up phone call and remote assessments took place 14 days after the last dose of treatment. Thereafter the subject continued the observational period as scheduled (Table 2).

If at any time during a follow-up period or the observational period, a subject decided to terminate the study, the subject contacted the site and completed their remote assessments as an early termination (ET) visit. An ET visit was on the same day as an EOT visit if a subject discontinued study drug and terminated the study on the same day during a treatment period; in this case, all events scheduled for the EOT visit was conducted.

A subject was deemed lost to follow-up after attempts at contacting the subject had been unsuccessful.

### Individual Subject Stopping Criteria

This was the first study in which longitudinal re-treatment with Compound 1 were examined. Based on known withdrawal symptoms with other GABAergic drugs and non-clinical findings in a 9-month study of Compound 1 in dogs (Investigator's Brochure), there was a potential for withdrawal-related events, including seizure. The following guidelines for study drug discontinuation or dose reduction were presented to support subject safety: (1) any subject reporting a confirmed or suspected seizure at any time was discontinued from treatment and was not be eligible for another treatment cycle, but was continued to be followed in the study; (2) Following the first treatment period, the Investigator monitored the course of CNS-based signs and symptoms suggestive of a seizure which were not accounted for by comorbid psychiatric or medical conditions. Examples of reported serious or severe events which may reflect an oncoming and/or increased risk for seizure included temporary confusion, tremors, involuntary muscle fasciculations or jerking movements of arms or legs, or paresthesia. Should such symptoms occur, the Investigator, in consultation with the Sage Medical Monitor, considered decreasing the dose of study drug to 20 mg, stopping treatment to assess the effect on the symptom(s) (e.g., resolution, improvement, etc.), or discontinuing the subject from treatment. A subject who discontinues treatment remained in the study and continue protocol-required assessments until the end of the study.

As this was an open-label study, any severe or serious events, were evaluated in an ongoing manner, including an evaluation of the benefit/risk profile of Compound 1 in the context of the current study. As a result, the Sponsor modified or discontinued the study.

Prior and Concomitant Medications and/or Supplements

The start and end dates, route, dose/units, frequency, and indication for all medications and/or supplements taken within 30 days prior to Screening and throughout the duration of the study were recorded. In addition, antidepressant therapies taken in the 3 years prior to Screening were recorded.

Any medication and/or supplement determined necessary for the welfare of the subject were given at the discretion of the Investigator at any time during the study.

Antidepressants that have been taken at the same dose for at least 60 days prior to Day 1 were permitted if the subject intended to continue the stable dose through the initial treatment and follow-up period (through Day 28 of Cycle 1).

See Table 3 for allowed concomitant psychotropic medications during each period of the study.

### Medication Use for Depressive Symptom Worsening Following a Compound 1 Treatment Cycle

For subjects achieving remission or response at Day 15 (78.6%), 6.1% had a HAM-D ≥22 at Day 42; another 18.2% had a HAM-D score of 16 to 21 at Day 42. This indicates that most subjects who may experience a new MDE will have this experience after they reach the minimal required period (8 weeks or 56 days) before a new Compound 1 treatment cycle. Because of this, most subjects were eligible (i.e., PHQ-9 ≥10 and HAM-D ≥20 confirmed over 2 weeks) for a Compound 1 treatment cycle when needed; a 2-week period was required to establish a new MDE (DSM-5).

For subjects who experienced worsening depressive symptoms after Day 28 and were not yet eligible for a new Compound 1 treatment cycle, there were 2 intervention options: as-needed medications (limited to a maximum of 4 days per week) and/or introduction of a new ADT or an increase in the dose of a current ADT (Table 3). To maintain equivalence in clinical status across all ADT use (i.e., new Compound 1, new ADT, or increase dose of current ADT), a requirement for PHQ-9 ≥10 and HAM-D ≥20 confirmed over 2 weeks was required in all ADT use conditions. If a subject on a stable ADT was experiencing worsening depressive symptoms (PHQ-9≥10), it was recommended that only as-needed medications would be used if the HAM-D score was <20; if the HAM-D score was ≥20, the current dose was increased or a new ADT was introduced. Furthermore, clinicians considered an individual subject's initial experience with Compound 1 when starting any new ADT, as it may substantially reduce the likelihood that the subject would be eligible for a new Compound 1 treatment cycle once time allows (i.e., HAM-D may be <20). There was no PHQ-9 or HAM-D score requirement for as-needed medication use.

Permitted as-needed medications for symptom management include benzodiazepines, GABA-modulators for insomnia (e.g., eszopiclone, zopiclone, zaleplon, and zolpidem), and non-GABA treatments for insomnia; use of such treatments should be limited to a maximum of 4 days per week.

If as-needed medications and/or a new ADT was introduced or the dose of a current ADT was increased and the subject continues to exhibit a HAM-D≥20, a new Compound 1 treatment cycle could be initiated at Day 70 or later. After completion of a new Compound 1 cycle, continued use of any intervention(s) used during the previous Observation Period was at the Investigator's discretion.

Any benzodiazepines and/or GABA-modulating medication use during the Observation Period was stopped 7 days prior to any new Compound 1 treatment cycle. As-needed non-GABA modulating medication use was discontinued 1 day prior to any new Compound 1 treatment cycle.

Medications intended for contraception were permitted for female subjects.

TABLE 3

| | | Psychotropic | |
|---|---|---|---|
| | | medications | |
| Period | Timing* | allowed | Rationale |
| Compound 1 | Day 1 to 14 | Compound 1 | Assess |
| Treatment | | Stable ADT | Compound |
| (e.g., | | No as-needed | 1 response |
| episodic | | medications^{a} | |
| dosing | | No new ADT | |
| regimen) | | | |
| Compound 1 | Day 15 to 28 | Stable ADT | Assess |
| Follow-up | | No as-needed | Compound |
| | | medications^{a} | 1 safety |
| | | No new ADT | in follow-up |
| Observation | Day 29 to 7 days | Benzodiazepines | Establish ‘full |
| | prior to next | (regular or as- | remission’ |
| | Compound 1 | needed) | Assess symptom |
| | treatment cycle, | As-needed GABA- | course over time |
| | if applicable | modulators | |
| | | for insomnia | |
| | Day 29 to 1 day | As-needed non- | |
| | prior to next | GABA-modulating | |
| | Compound 1 | treatments for | |
| | treatment cycle, | insomnia | |
| | if applicable | | |
| | Day 29 through | Stable ADT | |
| | next Compound 1 | New ADT (except | |
| | treatment cycle, | benzodiazepines)^{b} | |
| | if applicable | | |

^{a}As-needed medications (benzodiazepines, GABA-modulators for insomnia [eg, eszopiclone, zopiclone, zaleplon, and zolpidem], and non-GABA treatments for insomnia [eg melatonin, Benadryl [anti-histamines], trazodone, mirtazapine, etc]) should be limited to a maximum of 4 days per week.
^{b}If a subject on a stable ADT is experiencing worsening depressive symptoms (PHQ-9 ≥10), it is recommended that only as-needed medications be used if the HAM-D score is <20; if the HAM D score is ≥20, the current ADT dose may be increased or a new ADT may be introduced.
*Timing relative to the initial/previous cycle of Compound 1
ADT = antidepressant; Stable ADT = ADT started prior to study and continued at baseline or any new ADT started during an Observation Period and continued thereafter through a new Compound 1 cycle

### Example 3: A Phase 3, Randomized, Double-Blind, Placebo-Controlled Study of the Efficacy and Safety of Compound 1 with a Fixed, Repeated Treatment Regimen on Relapse Prevention in Adults with Major Depressive Disorder (MDD)

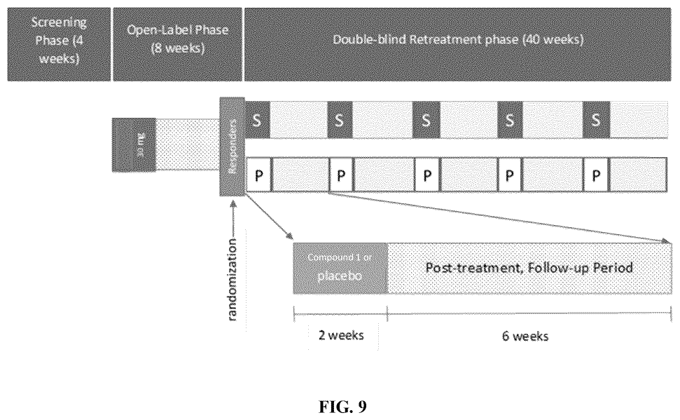

This was an open label phase followed by a randomized, double-blind, placebo-controlled phase study to assess the effect of Compound 1 monotherapy in a fixed, repeated treatment regimen versus placebo on relapse prevention in adult subjects with MDD (Montgomery-Åsberg Depression Rating Scale [MADRS] ≥32, HAM-D ≥22) that were not currently taking antidepressants. See FIG. 9 for a schematic of the study design.

The planned duration of subject participation was up to 52 weeks, including a Screening Period (up to 4 weeks), an Open-label (OL) Phase (8 weeks), and a Double-blind (DB) Phase (40 weeks).

The Screening Period (Table 4) began with the signature of the informed consent form (ICF); the ICF was signed prior to beginning any screening activities. The diagnosis of MDD was made according to Structured Clinical Interview for Diagnostic and Statistical Manual of Mental Disorders, Fifth Edition (DSM-5) Clinical Trial Version (SCID-5-CT) performed by a qualified healthcare professional. Subjects underwent preliminary screening procedures at the Screening Visit to determine eligibility, including completion of the MADRS and CGI-S.

Beginning on Day 1 of the OL Phase, eligible subjects self-administered a single dose of study drug once daily in the evening with food, on an outpatient basis, for 14 consecutive days. Practical options included taking Compound 1 within 1 hour of dinner or taking Compound 1 later in the evening with solid food. Subjects returned to the study center during the OL treatment and follow-up periods as outlined in Table 5.

Subjects who completed the OL Phase (through Day 56) with no significant tolerability issues as judged by the Investigator and who exhibited a HAM-D response, defined as a ≥50% reduction from baseline in HAM-D total score, at Visits 4, 6, 7, and 8 (see Table 5) were eligible for the DB Phase. One excursion of <50% reduction from baseline in HAM-D total score at Visit 6, 7, or 8 was permitted for eligibility to the DB Phase.

Beginning on Day 1 of the DB Phase, eligible subjects were randomized to receive 30 mg of Compound 1 or matching placebo in a 1:1 ratio. The 40-week DB Phase consisted of five 14-day treatment periods, each separated by a 6-week follow-up period; the end of each follow-up period coincided with the first visit of the next treatment period. During the 14-day treatment periods, subjects self-administered a single dose of study drug once daily in the evening with food, on an outpatient basis. Subjects returned to the study center during the DB treatment and follow-up periods as outlined in Table 5.

During the follow-up periods of the DB Phase, depressive symptoms were monitored every 7 days via remote PHQ-9; if the PHQ-9 score was ≥10, the subject returned to the site as soon as possible to be assessed by the clinician-administered HAM-D (Table 6). If the HAM-D was ≥18 at this visit, the subject returned to the site in 7 to 14 days to be reassessed by the HAM-D (Table 6); if the HAM-D remains ≥18, the subject was considered to have relapsed. A subject was considered to have relapsed upon any worsening of depression requiring hospitalization, any Investigator-determined risk of suicide, and/or any other clinically-relevant event not requiring hospitalization. Subjects who relapsed during the DB Phase, as determined by the Investigator, were terminated from the study upon completion of an early termination (ET) visit; if a subject was determined to have relapsed during a treatment period, the subject had an End of Treatment (EOT) visit as soon as possible, and an ET visit 7 days after the EOT visit. Final determination of relapse was made by an Independent Relapse Adjudication Committee (IRAC).

If at any time during the study, 30 mg of Compound 1 was not tolerated, as assessed by the occurrence of a severe AE judged by the Investigator to be related to study drug, the dose was reduced to 20 mg and continued for the remainder of the treatment period. Dose adjustments related to moderate AEs were at the discretion of the Investigator. Subsequent treatment periods began with the 30-mg dose, regardless of whether a subject required a dose adjustment in a previous treatment period. Subjects who could not tolerate the 20-mg dose at any time were terminated from the study upon completion of an EOT visit as soon as possible, and an ET visit 7 days later.

The primary objective of this study was to evaluate the efficacy of Compound 1 with a fixed, repeated treatment regimen in the prevention of relapse in subjects with major depressive disorder (MDD) who had responded to OL treatment with Compound 1.

The secondary objective of this study was to evaluate the long-term safety and tolerability of a fixed, repeated treatment regimen of Compound 1 up to 1 year.

Other objectives of this study were to evaluate the efficacy of Compound 1 with a fixed, repeated treatment regimen versus placebo on work and activity impairment and health-related quality of life in subjects with MDD and to assess the pharmacokinetics (PK) of Compound 1 using a population PK approach.

Primary endpoint of this study was time to first relapse during the DB Phase (days; from first dose of study drug in the DB Phase to relapse [date] during the DB Phase).

Secondary endpoints of this study were: percentage of subjects who relapse during the DB Phase, change from baseline in the 17-item HAM-D total score at the end of each 14-day treatment period in the DB Phase, HAM-D response at the end of each 14-day treatment period in the DB Phase, defined as a ≥50% reduction in HAM-D score from baseline, HAM-D remission at the end of each 14-day treatment period in the DB Phase, defined as HAM-D total score ≤7, CGI-I response, defined as “much improved” or “very much improved”, at the end of each 14-day treatment period in the DB Phase, change from baseline in Clinical Global Impression-Severity (CGI-S) score at the end of each 14-day treatment period in the DB Phase, change from baseline in 9-item Patient Health Questionnaire (PHQ-9) score at the end of each 14-day treatment period in the DB Phase, time to first relapse during the DB phase (days; from first dose of study drug in DB, phase to relapse [date] during the DB Phase) for subjects who achieved HAM-D remission in the OL Phase, and incidence and severity of treatment-emergent adverse events (TEAEs).

Other endpoints of this study were: changes from baseline in clinical laboratory measures, vital signs, and electrocardiograms (ECGs), changes from baseline in suicidal ideation and behavior using the Columbia Suicide Severity Rating Scale (C-SSRS); evaluation of withdrawal symptoms as measured by the Physician Withdrawal Checklist (PWC-20); PRO measure of work and activity impairment, as assessed by change from baseline in the Work Productivity and Activity Impairment Questionnaire (WPAI) Specific Health Problem V2.0 (absenteeism, presenteeism, overall work impairment, and overall activity impairment); PRO measure of health-related quality of life, as assessed by change from baseline in the 5-dimension, 5-level questionnaire developed by the EuroQol Group (EQ-5D-5L); PK parameters (e.g., clearance) and exposure estimates (e.g., area under the curve over a dosing interval, maximum plasma concentration) as assessed via population PK methods.

### Inclusion Criteria:

### Qualified Subjects Met all of the Following Criteria:

1. Subject had signed an ICF prior to any study-specific procedures being performed.
2. Subject was a male or female between 18 and 65 years of age, inclusive.
3. Subject was in good physical health and has no clinically significant findings, as determined by the Investigator, on physical examination, 12-lead ECG, or clinical laboratory tests.
4. Subject agreed to adhere to the study requirements.
5. Subject had a diagnosis of MDD as diagnosed by SCID-5-CT, with symptoms that have been present for at least a 4-week period.
6. Subject had had at least 1 prior major depressive episode (MDE) in the 5 years prior to Screening (not including the current episode).
7. Subject had a MADRS total score of ≥32 and a HAM-D total score of ≥22 at Screening and Day 1 (prior to dosing) of the Open-label Phase.
8. Subject was willing to delay start of any antidepressant, anxiolytic, insomnia, psychostimulant, or prescription opioid regimens until after study completion.
9. Subjects received psychotherapy must have been receiving therapy on a regular schedule for at least 60 days prior to Day 1.
10. Female subject agreed to use one of the following methods of highly effective contraception during participation in the study and for 30 days following the last dose of study drug, unless they were postmenopausal (defined as no menses for 12 months without an alternative medical cause and confirmed by follicular stimulation hormone [FSH] >40 mIU/mL), surgically sterile (hysterectomy or bilateral oophorectomy), or does not engage in sexual relations which carry a risk of pregnancy:
   Combined (estrogen and progestogen containing) oral, intravaginal, or transdermal hormonal contraception associated with inhibition of ovulation
   Oral, injectable, or implantable progestogen-only hormonal contraception associated with inhibition of ovulation
   Intrauterine device
   Intrauterine hormone-releasing system
   Bilateral tubal ligation/occlusion
   Vasectomized partner.
11. Male subject agreed to use an acceptable method of effective contraception for the duration of study and for 5 days after receiving the last dose of the study drug, unless the subject does not engage in sexual relations which carry a risk of pregnancy. Acceptable methods of effective contraception for males includes vasectomy or a condom with spermicide used together with highly effective female contraception methods if the female partner(s) was of child-bearing potential (see Inclusion Criteria #10 for acceptable contraception methods).
12. Male subject was willing to abstain from sperm donation for the duration of the study and for 5 days after receiving the last dose of the study drug.
13. Subject agreed to refrain from drugs of abuse and alcohol for the duration of the study.

### Exclusion Criteria:

Subjects Who Meet any of the Following Criteria were Disqualified from Participation in this Study:
1. Subject had attempted suicide associated with the current episode of MDD.
2. Subject had a recent history or active clinically significant manifestations of metabolic, hepatic, renal, hematological, pulmonary, cardiovascular, gastrointestinal, musculoskeletal, dermatological, urogenital, neurological, or eyes, ears, nose, and throat disorders, or any other acute or chronic condition that, in the Investigator's opinion, would limit the subject's ability to complete or participate in this clinical study.
3. A body mass index (BMI) ≤18 or ≥50 kg/m2 at Screening was exclusionary; a BMI of 40 to 49 kg/m2, inclusive, at Screening was subject to a broader evaluation of medical comorbidities (such as sleep apnea, COPD), concomitant medications, prior tolerability of sedating agents.
4. Subject had treatment-resistant depression, defined as persistent depressive symptoms despite treatment with adequate doses of antidepressants within the current major depressive episode (excluding antipsychotics) from two different classes for at least 4 weeks of treatment. Massachusetts General Hospital Antidepressant Treatment Response Questionnaire was used for this purpose.
5. Subject had had vagus nerve stimulation, electroconvulsive therapy, or has taken ketamine within the current major depressive episode.
6. Subject had taken antidepressants within 60 days prior to Day 1.
7. Subject was taking benzodiazepines, barbiturates, or GABAA modulators (e.g., eszopiclone, zopiclone, zaleplon, and zolpidem) at Day −28, or subject has been using these agents daily or near-daily (>4 times per week) for more than one year at Day −28.
8. Subject was taking any benzodiazepine or GABA modulator with a half-life of ≥48 hours (e.g., diazepam) from 60 days prior to Day 1.
9. Subject was taking non-GABA anti-insomnia medications (e.g., melatonin, Benadryl [antihistamines], trazodone) or first or second generation (typical/atypical) antipsychotics at Day −14.
10. Subject was taking psychostimulants (e.g., methylphenidate, amphetamine) or opioids, regularly or as-needed, at Day −28.
11. Subject had a known allergy to Compound 1, allopregnanolone, or related compounds.
12. Subject had a positive pregnancy test at screening or on Day 1 prior to dosing.
13. Subject who was breastfeeding at Screening or on Day 1 (prior to administration of study drug) does not agree to temporarily cease giving breast milk to child (ren) from just prior to receiving study drug on Day 1 until 7 days after the last dose of study drug in each treatment period.
14. Subject had detectable hepatitis B surface antigen, anti-hepatitis C virus (HCV) and positive HCV viral load, or human immunodeficiency virus (HIV) antibody at screening.
15. Subject had a clinically significant abnormal 12-lead ECG at the screening or baseline visits. NOTE: mean QT interval calculated using the Fridericia method (QTcF) of >450 msec in males or >470 msec in females were the basis for exclusion from the study.
16. Subject had active psychosis per Investigator assessment.
17. Subject had a medical history of seizures.
18. Subject had a medical history of bipolar disorder, schizophrenia, and/or schizoaffective disorder.
19. Subject had a history of mild, moderate, or severe substance use disorder (including benzodiazepines) diagnosed using DSM-5 criteria in the 12 months prior to screening.
20. Subject had had exposure to another investigational medication or device within 30 days prior to screening.
21. Subject had previously participated in a Compound 1 or brexanolone clinical trial.
22. Subject had used any known strong inhibitors of cytochrome P450 (CYP) 3A4 within 28 days or 5 half-lives (whichever was longer) prior to the first dose of study drug or plans to use these during any treatment period, or consumed grapefruit juice, grapefruit, or Seville oranges, or products containing these within 14 days prior to the first dose of study drug for any treatment period or plans to consume these products during any treatment period.
23. Use of the following strong CYP3A inducers within 28 days prior to the first dose of study drug for any Compound 1 treatment period: rifampin, carbamazepine, enzalutamide, mitotane, phenytoin, and St John's Wort.
24. Subject had a positive drug and/or alcohol screen at screening or on Day 1 prior to dosing in the Open-label Phase.
25. Subject planned to undergo elective surgery or procedure requiring general anesthesia at any time from Screening through the duration of the study. Procedures requiring conscious sedation and ambulatory procedures performed under local anesthesia may be scheduled under the following guidelines:
   Procedures requiring conscious sedation (e.g. colonoscopy) no later than 7 days prior to the start of the first dose of each treatment period and no earlier than 7 days after the last dose of each treatment period from screening throughout the duration of the study.
   Elective ambulatory procedures performed under local anesthesia were allowed at any time during the study.
26. Subject had been diagnosed with and/or treated for any type of cancer (excluding basal cell carcinoma and melanoma in situ) within the past year prior to Screening.
27. Subject had had gastric bypass surgery, has a gastric sleeve or lap band, or has had any related procedures that interfere with gastrointestinal transit.
28. Subject regularly participated in night shift work or expected to perform night shift work during any 14-day treatment period (occasional night shift work during follow-up periods is permitted).

### Dosage and Mode of Administration

Compound 1 was available as hard gelatin capsules containing a white to off-white powder. In addition to Compound 1 Drug Substance, the Compound 1 capsules contained croscarmellose sodium, mannitol, silicified microcrystalline cellulose (SMCC), colloidal silicon dioxide and sodium stearyl fumarate as excipients. Colloidal silicon dioxide was either a component of the SMCC or a standalone excipient in the formulation. Compound 1 capsules were orally administered as a 30-mg or 20-mg dose.

### Reference Therapy, Dosage and Mode of Administration:

In the DB Phase, placebo was provided as hard gelatin capsules for oral administration in the evening with food.

### Duration of Treatment:

All subjects received a daily dose of Compound 1 from Day 1 through Day 14 in the OL Phase. Subjects that exhibited a HAM-D response to Compound 1 in the OL Phase were randomized to receive either daily doses of Compound 1 or placebo in 14-day treatment periods, separated by 6-week follow-up periods, for 40 weeks in the DB Phase (for a total of six 14-day treatment periods during the 52-week study).

TABLE 4

| | Screening |
|---|---|
| | Period |
| Study Day | −28 to −1 |
| Visit | 1 |
| Study Procedure | |
| Informed Consent | X |
| Duplicate Subject Check (US only)^{a} | X |
| Inclusion/Exclusion | X |
| Demographics | X |
| Medical/Family History | X |
| SCID-5 | X |
| ICD-10^{b} | X |
| MGH-ATRQ | X |
| Serum FSH test^{c} | X |
| Full Physical Examination^{d} | X |
| Body Weight/Height | X |
| Clinical Laboratory Assessments^{e} | X |
| Drug & Alcohol Screen^{f} | X |
| Serum Pregnancy Test | X |
| Hepatitis & HIV Screen | X |
| Subject training^{g} | X |
| Vital Signs^{h} | X |
| 12-Lead ECGⁱ | X |
| Baseline/Screening C-SSRS | X |
| HAM-D^{j} | X |
| MADRS | X |
| CGI-S | X |
| Adverse Events/SAEs^{k} | X |
| Prior Medications | X |

CGI-S—Clinical Global Impression - Severity; C-SSRS = Columbia Suicide Severity Rating Scale; ECG = electrocardiogram; FSH = follicle stimulating hormone; HAM-D = Hamilton Rating Scale for Depression, 17-item; HIV = human immunodeficiency virus; ICD-10 = International Statistical Classification of Diseases and Related Health Problems version 10; MADRS = Montgomery-Åsberg Depression Rating Scale; MGH ATRQ = Massachusetts General Hospital Antidepressant Treatment Response Questionnaire; SCID-5 = Structured Clinical Interview for Diagnostic and Statistical Manual of Mental Disorders, Fifth Edition; SAE = serious adverse event; US = United States
^{a}Subjects at US sites will be asked to authorize that their unique subject identifiers be entered into a registry with the intent of identifying subjects who may meet exclusion criteria for participation in another clinical study.
^{b}ICD-10 codes to be collected if available.
^{c}A serum FSH test will be conducted at Screening for female subjects that are not surgically sterile to confirm whether a female subject with ≥12 months of spontaneous amenorrhea meets the protocol-defined criteria for being post-menopausal.
^{d}A full physical examination will be conducted, including assessment of body systems (eg, head, eye, ear, nose, and throat; heart; lungs; abdomen; and extremities).
^{e}Clinical laboratory tests will include hematology, serum chemistry, coagulation, and urinalysis.
^{f}Urine toxicology for selected drugs of abuse (as per the laboratory manual) and breath test for alcohol.
^{g}Subjects will be trained on use of software applications and devices necessary for the conduct of the study by site personnel.
^{h}Vital signs include oral temperature (° C.), respiratory rate, heart rate, and blood pressure (supine and standing). Heart rate and blood pressure to be collected in supine position at all scheduled time points after the subject has been resting for 5 minutes and then after approximately 3 minutes in the standing position. Vital signs may be repeated at the discretion of the Investigator as clinically indicated.
ⁱTriplicate ECGs will be collected.
^{j}The HAM-D is to be completed as early during the visit as possible. The assessment timeframe for HAM-D scales will refer to the past 7 days (1 week).
^{k}Adverse events will be collected starting at the time of informed consent and throughout the duration of the subject's participation in the study.

TABLE 5

| | | | | | | | Double-blind | | | | Double-blind | | | |
|---|---|---|---|---|---|---|---|---|---|---|---|---|---|---|
| | Open-Label | | | | | | Period 1 | | | | Period 2 | | | |
| | Study Day | | | | | | | | | | | | | |
| | | 8 | 15 | 21 | 28 | 42 | 56 | 63 | 70 | 76 | 111 | 118 | 125 | 131 |
| | 1 | (+1) | (+1) | (±1) | (±1) | (±1) | (±1) | (+1) | (+1) | (±3) | (±3) | (+1) | (+1) | (±3) |
| | Visit | | | | | | | | | | | | | |
| | 2 | 3 | 4 | 5 | 6 | 7 | 8 | 9 | 10 | 11 | 12 | 13 | 14 | 15 |
| | Treatment Period Day | | | | | | | | | | | | | |
| Study | | | 15/ | | | | 56/ | | 15/ | | 56/ | | 15/ | |
| Procedure | 1 | 8 | EOT^{a} | 21 | 28 | 42 | 1^{b} | 8 | EOT^{a} | 21 | 1 | 8 | EOT^{a} | 21 |
| Inclusion/ | X | | | | | | | | | | | | | |
| Exclusion | | | | | | | | | | | | | | |
| MADRS | X | | | | | | | | | | | | | |
| Subject | X | | | | | | | | | | | | | |
| training^{c} | | | | | | | | | | | | | | |
| Randomization | | | | | | | X | | | | | | | |
| Abbreviated | X | | | X | | | X | | | X | X | | | X |
| Physical | | | | | | | | | | | | | | |
| Examination | | | | | | | | | | | | | | |
| Body Weight | X | | X | | | | X | | X | | X | | X | |
| Clinical | X | | X | | | | X | | X | | X | | | |
| Laboratory | | | | | | | | | | | | | | |
| Assessments^{d} | | | | | | | | | | | | | | |
| Drug & | X | | | | | | X | | | | X | | | |
| Alcohol | | | | | | | | | | | | | | |
| Screen^{e} | | | | | | | | | | | | | | |
| Urine | X | | X | | | | X | | X | | X | | X | |
| Pregnancy Test | | | | | | | | | | | | | | |
| Vital Signs^{f} | X | X | X | X | X | X | X | X | X | X | X | X | X | X |

| | Double-blind | | | | Double-blind | | | | Double-blind | | | | |
|---|---|---|---|---|---|---|---|---|---|---|---|---|---|
| | Period 3 | | | | Period 4 | | | | Period 5 | | | | EOS |
| | Study Day | | | | | | | | | | | | |
| | 166 | 173 | 180 | 186 | 221 | 228 | 235 | 241 | 276 | 283 | 290 | 296 | 331 |
| | (±3) | (+1) | (+1) | (±3) | (±3) | (+1) | (+1) | (±3) | (±3) | (+1) | (+1) | (±3) | (±3) |
| | Visit | | | | | | | | | | | | |
| | 16 | 17 | 18 | 19 | 20 | 21 | 22 | 23 | 24 | 25 | 26 | 27 | 28 |
| | Treatment Period Day | | | | | | | | | | | | |
| Study | 56/ | | 15/ | | 56/ | | 15/ | | 56/ | | 15/ | | 56/ |
| Procedure | 1 | 8 | EOT^{a} | 21 | 1 | 8 | EOT^{a} | 21 | 1 | 8 | EOT^{a} | 21 | ET^{a} |
| Inclusion/ | | | | | | | | | | | | | |
| Exclusion | | | | | | | | | | | | | |
| MADRS | | | | | | | | | | | | | |
| Subject | | | | | | | | | | | | | |
| training^{c} | | | | | | | | | | | | | |
| Randomization | | | | | | | | | | | | | |
| Abbreviated | X | | | X | X | | | X | X | | | X | X |
| Physical | | | | | | | | | | | | | |
| Examination | | | | | | | | | | | | | |
| Body Weight | X | | X | | X | | X | | X | | X | | X |
| Clinical | X | | X | | X | | | | X | | X | | X |
| Laboratory | | | | | | | | | | | | | |
| Assessments^{d} | | | | | | | | | | | | | |
| Drug & | X | | | | X | | | | X | | | | |
| Alcohol | | | | | | | | | | | | | |
| Screen^{e} | | | | | | | | | | | | | |
| Urine | X | | X | | X | | X | | X | | X | | X |
| Pregnancy Test | | | | | | | | | | | | | |
| Vital Signs^{f} | X | X | X | X | X | X | X | X | X | X | X | X | X |

| | | | | | | | Double-blind | | | | Double-blind | | | |
|---|---|---|---|---|---|---|---|---|---|---|---|---|---|---|
| | Open-Label | | | | | | Period 1 | | | | Period 2 | | | |
| | Study Day | | | | | | | | | | | | | |
| | | 8 | 15 | 21 | 28 | 42 | 56 | 63 | 70 | 76 | 111 | 118 | 125 | 131 |
| | 1 | (+1) | (+1) | (±1) | (±1) | (±1) | (±1) | (+1) | (+1) | (±3) | (±3) | (+1) | (+1) | (±3) |
| | Visit | | | | | | | | | | | | | |
| | 2 | 3 | 4 | 5 | 6 | 7 | 8 | 9 | 10 | 11 | 12 | 13 | 14 | 15 |
| | Treatment Period Day | | | | | | | | | | | | | |
| Study | | | 15/ | | | | 56/ | | 15/ | | 56/ | | 15/ | |
| Procedure | 1 | 8 | EOT^{a} | 21 | 28 | 42 | 1^{b} | 8 | EOT^{a} | 21 | 1 | 8 | EOT^{a} | 21 |
| 12-Lead ECG^{g} | X | | X | | | | X | | X | | X | | | |
| C-SSRS^{h} | X | X | X | X | X | X | X | X | X | X | X | X | X | X |
| HAM-Dⁱ | X | X | X | | X | X | X | X | X | | X | X | X | |
| CGI-S | X | X | X | | X | X | X | X | X | | X | X | X | |
| CGI-I | | X | X | | X | X | X | X | X | | X | X | X | |
| WPAI | X | | X | | | X | X | X | | | X | X | | |
| EQ-5D-5L | X | | X | | | | X | X | X | | X | X | X | |
| PWC-20 | | | X | X | | | | | X | X | | | X | X |
| Plasma PK^{j} | | X | X | | | | | X | X | | | X | X | |
| Study Drug | X | X | | | | | X | X | | | X | X | | |
| Dispensation | | | | | | | | | | | | | | |
| Study Drug | X (QD for | | | | | | X (QD for | | | | X (QD for | | | |
| Administration | 14 days) | | | | | | 14 days) | | | | 14 days) | | | |
| Study Drug | | X | X | | | | | X | X | | | X | X | |
| Accountability | | | | | | | | | | | | | | |
| Return | | | | | | | | | | | | | | |

| | |
|---|---|
| PHQ-9^{k} | X (QW) |
| Adverse | X |
| Events/SAEs^{l} | |
| Concomitant | X |
| Medications | |

| | Double-blind | | | | Double-blind | | | | Double-blind | | | | |
|---|---|---|---|---|---|---|---|---|---|---|---|---|---|
| | Period 3 | | | | Period 4 | | | | Period 5 | | | | EOS |
| | Study Day | | | | | | | | | | | | |
| | 168 | 173 | 180 | 186 | 221 | 228 | 235 | 241 | 276 | 283 | 290 | 296 | 331 |
| | (+3) | (+1) | (+1) | (±3) | (+3) | (+1) | (+1) | (±3) | (±3) | (+1) | (+1) | (±3) | (±3) |
| | Visit | | | | | | | | | | | | |
| | 16 | 17 | 18 | 19 | 20 | 21 | 22 | 23 | 24 | 25 | 26 | 27 | 28 |
| | Treatment Period Day | | | | | | | | | | | | |
| Study | 56/ | | 15/ | | 56/ | | 15/ | | 56/ | | 15/ | | 56/ |
| Procedure | 1 | 8 | EOT^{a} | 21 | 1 | 8 | EOT^{a} | 21 | 1 | 8 | EOT^{a} | 21 | ET^{a} |
| 12-Lead ECG^{g} | X | | X | | X | | | | X | | X | | X |
| C-SSRS^{h} | X | X | X | X | X | X | X | X | X | X | X | X | X |
| HAM-Dⁱ | X | X | X | | X | X | X | | X | X | X | | X |
| CGI-S | X | X | | | X | X | X | | X | X | X | | X |
| CGI-I | X | X | X | | X | X | X | | X | X | X | | X |
| WPAI | X | X | | | X | X | | | X | X | | | X |
| | | | | | | | | | | | | | (ST |
| | | | | | | | | | | | | | only) |
| EQ-5D-5L | X | X | X | | X | X | X | | X | X | X | | X |
| PWC-20 | | | X | X | | | X | X | | | X | X | X |
| Plasma PK^{j} | | X | X | | | X | X | | | X | X | | X |
| | | | | | | | | | | | | | (ST |
| | | | | | | | | | | | | | only) |
| Study Drug | X | X | | | X | X | | | X | X | | | |
| Dispensation | | | | | | | | | | | | | |
| Study Drug | X (QD for | | | | X (QD for | | | | X (QD for | | | | |
| Administration | 14 days) | | | | 14 days) | | | | 14 days) | | | | |
| Study Drug | | X | X | | | X | X | | | X | X | | |
| Accountability | | | | | | | | | | | | | |
| Return | | | | | | | | | | | | | |

| | |
|---|---|
| PHQ-9^{k} | X (QW) |
| Adverse | X |
| Events/SAEs^{l} | |
| Concomitant | X |
| Medications | |

CGI-I = Clinical Global Impression - Improvement; CGI-S—Clinical Global Impression - Severity; C-SSRS = Columbia Suicide Severity Rating Scale; ECG = electrocardiogram; EQ-5D-5L = EuroQol Group 5-dimension, 5-level questionnaire; EOS = End of study; ET = Early termination; HAM-D = Hamilton Rating Scale for Depression, 17-item; QD = Once daily; QW = Once weekly; PHQ-9 = 9-item Patient Health Questionnaire; PWC-20 = 20-item Physical Withdrawal Checklist; SAE = serious adverse event; WPAI = Work Productivity and Activity Impairment Questionnaire
^{a}Subjects who discontinue treatment early should return to the site for an end of treatment (EOT) visit as soon as possible, preferably the day after treatment is discontinued. Follow-up visits should occur as scheduled relative to the list day of treatment. If at any time after the EOT visit, a subject decides to terminate the study, the subject should return for an early termination (ET) visit. The EOT and ET visits can be on the same day if a subject discontinues study drug and terminates the study on the same day during a clinic visit; in this case, all events scheduled for the EOT visit will be conducted.
^{b}The completion of the Open-label Phase coincides with the first day of the Double-Blind Phase (Study Day 56, Visit 7). Subjects that do not exhibit a response to SAGE-217 in the Open-label Phase (see criteria above) will be terminated from the study on this day.
^{c}Subjects will be trained on use of software applications and devices necessary for the conduct of the study by site personnel.
^{d}Clinical laboratory tests will include hematology, serum chemistry, coagulation, and urinalysis.
^{e}Urine toxicology for selected drugs of abuse (as per the laboratory manual) and breath test for alcohol.
^{f}Vital signs include oral temperature (° C.), respiratory rate, heart rate, and blood pressure (supine and standing). Heart rate and blood pressure to be collected in supine position at all scheduled time points after the subject has been resting for 5 minutes and then after approximately 3 minutes in the standing position. Vital signs may be repeated at the discretion of the Investigator as clinically indicated.
^{g}Triplicate ECGs will be collected. When ECGs and PK sample collection occur on the same day, the 12-lead ECGs will be performed before PK sample collection.
^{h}The “Since Last Visit” C-SSRS form will be completed.
ⁱThe HAM-D is to be completed as early during the visit as possible. The assessment timeframe for HAM-D scales will refer to the past 7 days (1 week) at Day 56/1 of the Double-blind Phase, and “Since Last Visit” for Day 1 of the Open-Label Phase and all other visits.
^{j}Plasma samples for PK analysis will be collected anytime during the clinic visit. The date and time of sample collection and date and time of the last dose administration must be recorded. When ECGs and PK sample collection occur on the same day, the 12-lead ECGs will be performed before PK sample collection.
^{k}All PHQ-9 assessments will be performed via a mobile phone-supported software application. The subject will take the PHQ-9 every 7 days; if the PHQ-9 score is ≥10, the subject will return to the site as soon as possible to be assessed by the clinician-administered HAM-D. If the HAM-D is ≥18 at this visit, the subject will return to the site in 7 to 14 days to be reassessed by the HAM-D. See Table 3 for the assessments to be conducted at these visits.
^{l}Adverse events will be collected starting at the time of informed consent and throughout the duration of the subject's participation in the study.

TABLE 6
Study Procedure

| | |
|---|---|
| Abbreviated Physical Examination | X |
| Clinical Laboratory Assessments^{a} | X |
| Urine Pregnancy Test | X |
| Vital Signs^{b} | X |
| C-SSRS^{c} | X |
| HAM-D^{d} | X |
| CGI-S | X |
| CGI-I | X |
| WPAI | X |
| EQ-5D-5L | X |
| Adverse Events/SAEs^{e} | X |
| Concomitant Medications | X |

CGI-I = Clinical Global Impression - Improvement; CGI-S—Clinical Global Impression - Severity; C- SSRS = Columbia Suicide Severity Rating Scale; ECG = electrocardiogram; EQ-5D-5L = EuroQol Group 5-dimension, 5-level questionnaire; HAM-D = Hamilton Rating Scale for Depression, 17-item; SAE = serious adverse event; WPAI = Work Productivity and Activity Impairment Questionnaire
^{a}Clinical laboratory tests will include hematology, serum chemistry, coagulation, and urinalysis.
bVital signs include oral temperature (° C.), respiratory rate, heart rate, and blood pressure (supine and standing). Heart rate and blood pressure to be collected in supine position at all scheduled time points after the subject has been resting for 5 minutes and then after approximately 3 minutes in the standing position. Vital signs may be repeated at the discretion of the Investigator as clinically indicated.
^{c}The “Since Last Visit” C-SSRS form will be completed,
^{d}The HAM-D is to be completed as early during the visit as possible. The assessment timeframe for HAM-D scales will refer to “Since Last Visit”.
^{e}Adverse events will be collected starting at the time of informed consent and throughout the duration of the subject's participation in the study.

### Example 4

Cognitive deficits can occur in individuals with depression and anxiety, for example major depressive disorder (MDD). Subjects receiving Compound 1 will be assessed using a battery of cognition tests, or Cogstate tests for changes, if any, in cognition.

Cogstate tests can be designed to measure specific areas of cognition, and can be grouped together to form customized batteries based on the unique requirements of the study design and population. Examples of Cogstate tests are as follows:

The Behavioral Pattern Separation Object test measures recognition memory using photos of objects. The participant is presented with a series of photos of common objects and must decide whether each object is used indoors or outdoors. The participant is then presented with a photo of an object and must recall whether the object is the same, similar or different to the photos they have already been shown.

The Continuous Paired Associate Learning test measures visual memory using a paired associative learning paradigm. In this test, the participant must learn and remember the pictures hidden beneath different locations on the screen. In the first stage of the test the pre-test on-screen instructions ask: “In what locations do these pictures belong”. A picture is presented in the centre of the screen. The participant taps the peripheral location of the picture and must remember its location. During the second stage of the test the same pictures are presented in the centre of the screen, however the peripheral location of each picture is hidden. The participant must tap on the peripheral location where the picture previously appeared.

The Detection test measures processing speed using a simple reaction time paradigm. The on-screen instructions ask: “Has the card turned over?”. A playing card is presented face down in the center of the screen. The card flips over so it is face up. As soon as the card flips over the participant must press “Yes”. The participant is encouraged to work as quickly as they can and be as accurate as possible.

The Face Name Associative Memory Exam measures associative memory using photos of real-life faces. The participant is presented with a series of facial photos and names, with each face paired with a name. The participant must remember the face-name pair.

The Go-No Go Test is a measure of response inhibition and uses a well-validated recognition reaction time paradigm with playing card stimuli. In this test, the playing cards are all either red or black jokers. The subject is asked whether the card displayed in the center of the screen is black. The subject is to press the Yes key when the joker card is black and to withhold a response (i.e., not respond) when it is red.

The Groton Maze Learning Test measures executive function using a maze learning paradigm. A 10×10 grid of tiles is presented to the participant on the screen. A 28-step pathway is hidden among these tiles. A blue tile indicates the start and a tile with red circles indicates the finish. The participant must move one step at a time from the start toward the end by touching a tile next to their current location. If the correct move is made a green checkmark appears and if the move is incorrect a red cross is revealed. Once completed, they are returned to the start location to repeat the test and must try to remember the pathway they have just completed.

The Identification test measures attention using a choice reaction time paradigm. The on-screen instructions ask: “Is the card red?”. A playing card is presented face down in the center of the screen. The card flips over so it is face up. As soon as it flips over the participant must decide whether the card is red or not. If it is red the participant should press “Yes”, and if it is not red the participant should press “No”. The participant is encouraged to work as quickly as they can and be as accurate as possible.

The International Shopping List Test measures verbal learning using a word list learning paradigm. The participant is read a shopping list and must remember and recall as many items from the list as possible.

The One Back test measures working memory using an n-back paradigm. The on-screen instructions ask: “Is the previous card the same?”. A playing card is presented face up in the center of the screen. The participant must decide whether the card is the same as the previous card. If the card is the same the participant should press “Yes”, and if it is not the same the participant should press “No”. The participant is encouraged to work as quickly as they can and be as accurate as possible.

The One Card Learning test measures visual memory using a pattern separation paradigm. The on-screen instructions ask: “Have you seen this card before in this test?”. A playing card is presented face up in the center of the screen and the participant must decide whether they have seen the card before in this test. The participant is encouraged to work as quickly as they can and be as accurate as possible.

The Set-Shifting test uses a set shifting paradigm to measure executive function. The on-screen instructions ask: “Is this a target card?”. A playing card is presented face up in the center of the screen with the word “Number” or “Color” above it. If the word is “Color” the participant must guess whether the target card is black or red. If the word is “Number” the participant must guess whether the current number displayed on the card is correct. At the beginning of the test, the participant simply needs to guess whether the current card is the target card. If they think the card is the target card, the participant should press “Yes”. If they think the card is not the target card, they must press “No”. As the participant makes their guesses, feedback is provided and the next card is not displayed until a correct response has been made. Once the participant has made their way through a set of cards the hidden rule changes (e.g., from one color to the other color [intra-dimensional shift], or from color to number [extra-dimensional shift]). The participant is not told when these set-shifts occur, and they must learn the new target rule to proceed through the test. The participant is encouraged to work as quickly as they can and be as accurate as possible.

The Social-Emotional Cognition Test measures emotional recognition using an odd-man out paradigm. The on-screen instructions ask: “Tap the odd one out”. Four pictures are presented on the screen. One of these pictures will be different to the others and the participant must decide which picture is different and tap that picture. The participant is encouraged to work as quickly as they can and be as accurate as possible.

The Two Back test measures working memory using an n-back paradigm. The on-screen instructions ask: “Is the card the same as that shown two cards ago?”. A playing card is presented face up in the center of the screen. The participant must decide whether the card is the same as the card shown two cards previously. If the card is the same the participant should press “Yes”, and if it is not the same the participant should press “No”. The participant is encouraged to work as quickly as they can and be as accurate as possible.

To assess cognitive decline, deficits, or improvements in subjects receiving Compound 1, a battery of tests may be used to assess cognition, such as the battery presented in Table 7.

TABLE 7
Details of the computerized cognitive tests in the Cogstate battery.

| | | | | |
|---|---|---|---|---|
| | | | Main | |
| | | Theoretical | cognitive | References |
| | Test | cognitive | domain | demonstrating |
| Cogstate test | requirements | model | assessed | validity |

RECOMMENDED TESTS

| | | | | |
|---|---|---|---|---|
| Detection Test | Respond as | Simple | Psychomotor | 1-3 |
| | quickly as possible | reaction | function | |
| | to change in visual | time | | |
| | stimulus | | | |
| Identification Test | Decide as quickly | Choice | Visual | 1-3 |
| | as possible about | reaction | attention | |
| | the physical | time | | |
| | characteristic of a | | | |
| | visual stimulus | | | |
| International | Learn and recall a | Verbal list | Verbal | 2, 4-6 |
| Shopping List | set of 12 words | learning | episodic | |
| Test with Delayed | across three trials, | and | memory | |
| Recall | then recall this list | delayed | | |
| | after a delay | recall | | |
| Groton Maze | Find and learn a | Hidden | Executive | 2, 4-6 |
| Learning Test | pathway hidden | pathway | function | |
| | beneath a 10 × 10 | maze | | |
| | matrix of identical | learning | | |
| | tiles | | | |

Subjects may be assessed with a battery of Cogstate tests before administration of Compound 1, during the administration of Compound 1, and after the administration of Compound 1.

### REFERENCES CITED IN TABLE 7

1 Davis M T, DellaGiola N, Matuskey D, et al. Preliminary evidence concerning the pattern and magnitude of cognitive dysfunction in major depressive disorder using cogstate measures. J Affect Disord. 2017; 218. doi:10.1016/J.jad.2017.04.064
2. Holmes S E, Scheinost D, Finnerna S J, et al. Lower synaptic density is associated with depression severity and network alterations. Nat Commun. 2019; 10 (1): 1529. doi:10.1038/s41467-019-09562-7
3. Olver J S, Ignatiadis S, Maruff P, Burrows G D, Norman T R. Quetiapine mentation in depressed patients with partial response to antidepressants. Hum Psychopharmacol. 2008; 23(8):663-660. doi:10.1002/hup.970
4. Gálvez V, Li A, Huggins C, et al. Repeated intranasal ketamine for treatment-resistant depression—the way to go? Results from a pilot randomised controlled trial. 2018. doi:10.1177/0269881118760660
5. Hashimoto K, Yoshida T, Ishikawa M, et al. Increased serum levels of serine enantiomers in patients with depression. Acta Neuropsychiatr. 2015:(November):6. doi:10.1017/neu.2015.59
6. Yoshida T, Ishikawa M, Nillsu T, et al. Decreased serum levels of mature brain-derived neurotrophic factor (BDNF), but not its precursor proBDNF, in patients with major depressive disorder. PLOS One. 2012; 7 (8): e42676. doi:10.1371/journal.pone.0042676

### Example 5. Results of a Phase 3 Study of Compound 1 in Major Depressive Disorder (MDD)

Compound 1 was evaluated in a double-blind, randomized placebo-controlled Phase 3 study in Major Depressive Disorder (MDD). The evaluated the efficacy, safety and pharmacokinetics of Compound 1 in adult patients diagnosed with MDD (MADRS total score ≥32 and a HAM-D total score ≥22).

Compound 1 was administered to a patient, either in 20 mg daily doses once a day for 14 days or 30 mg once a day for 14 days.

At Day 15, the primary endpoint and end of dosing, patients randomized to Compound 1 (30 mg) demonstrated a reduction in depressive symptoms of 12.6 in the HAM-D total score compared with 11.2 in patients who received placebo (LS Mean Difference from placebo −1.4, p=0.115).

Rapid onset of effect for Compound 1 (30 mg) (n=166) was noted beginning at Day 3 and statistical significance from placebo (n=157) was noted at all visits during the treatment period leading up to Day 15 (LS Mean Difference from placebo, p-value): Day 3 (−1.6, p=0.016), Day 8 (−2.1, p=0.008) and Day 12 (−2.1, p=0.018).

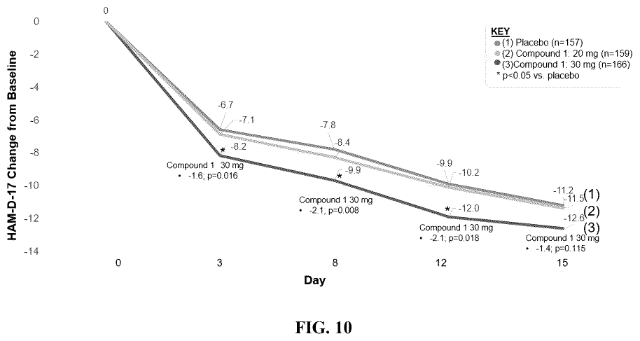

Plots of LS mean changes from baseline to Day 15 are shown in FIG. 10.

Improvements in depressive symptoms were sustained in all treatment groups through Day 42 of the double-blind portion of the study. Change from baseline in HAM-D total score at Day 42 for Compound 1 (30 mg) was −11.9 and for placebo was −11.7 (LS Mean Difference from placebo −0.5, p=0.807). Preliminary data suggest maintenance of improvement in depressive symptoms in those patients who have completed long-term follow-up up to 6 months. These data will continue to be collected in the coming months.

The 20 mg dose of Compound 1 did not separate from placebo in this dose-ranging study.

### Effect on Depressive Symptoms by Performance Factors

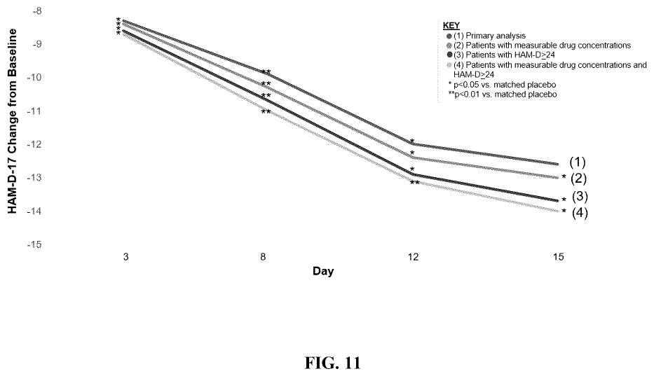

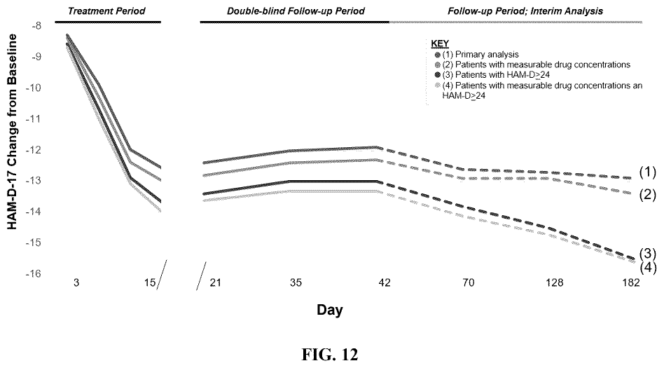

Post-hoc analyses were conducted to evaluate the effects of performance factors of Compound 1 in the primary analysis, in patients with measurable drug concentrations, in patients with HAM-D≥24, and in patients with measurable drug concentrations and HAM-D≥24, and including the primary outcome at Day 15 (FIG. 11). Changes in HAM-D score during double-blind follow up period and follow-period/interim analysis for these post-hoc analyses are further displayed in FIG. 12.

Change from baseline in HAM-D total score at Day 15, Compound 1 (30 mg) vs. placebo: Patients with Compound 1 measurable drug concentrations (n=151) (excluding 30 mg patients with no measurable drug concentration consistent with noncompliance): Compound 1 (30 mg) (−13.0) vs. placebo (−11.2); LS Mean Difference-1.8, p=0.048; patients with HAM-D≥24 (n=124): Compound 1 (30 mg) (−13.7) vs. placebo (−11.4); LS Mean Difference −2.3, p=0.032; and patients with Compound 1 measurable drug concentrations and HAM-D≥24 (n=115): Compound 1 (30 mg) (−14.0) vs. placebo (−11.4); LS Mean Difference-2.6; p=0.017.

### Safety and Tolerability

Compound 1 was generally well tolerated in the trial. The overall incidence of patients who experienced AEs during the 14-day treatment period and 28-day follow up was 54.2% for Compound 1 (30 mg), 50.0% for Compound 1 (20 mg) and 48.9% for placebo. Two patients receiving Compound 1 (30 mg) experienced serious adverse events (SAEs) during treatment: one suicide attempt on Day 5 in a patient with a longstanding history of MDD and a past suicide attempt, and one report of a bile duct stone after Day 2 requiring removal in a patient with a prior bile duct repair. In addition, three patients, one in each treatment group, reported SAEs during follow-up, all occurring at least one week following cessation of treatment: syncope and associated injuries which occurred with dehydration and orthostatic hypotension during exercise in a patient with a history of bradycardia (Compound 1 (30 mg), Day 28), multiple SAEs related to medical complications of cocaine ingestion (Compound 1 (20 mg), Day 39) and suicidal ideation (placebo, Day 22). The number of subjects having treatment emergent AEs leading to study drug discontinuation were similar in each treatment group (Compound 1 (30 mg) 2.1%, Compound 1 (20 mg) 1.6% and placebo 3.2%).

The most common adverse effects (AEs) (>5%) in any group (Compound 1 (30 mg), Compound 1 (20 mg) and placebo) during the 14-day treatment period and the 28-day follow up were: Headache (30 mg 6.3%, 20 mg 11.2%, placebo 7.4%); Dizziness (30 mg 5.7%, 20 mg 7.4%, placebo 3.7%); Somnolence (30 mg 6.8%, 20 mg 5.9%, placebo 4.2%); Fatigue (30 mg 6.8%, 20 mg 1.6%, placebo 2.6%); Diarrhea (30 mg 6.3%, 20 mg 5.9%, placebo 5.3%); Sedation (30 mg 4.7%, 20 mg 5.9%, placebo 3.2%); and Nausea (30 mg 3.6%, 20 mg 5.3%, placebo 4.7%). There were no AEs of loss of consciousness. There was no signal for increased suicidal ideation or suicidal behavior compared to baseline, as measured by Columbia Suicide Severity Rating Scale (C-SSRS).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

We claim:

1-20. (canceled)

21. A method for assessing cognitive decline, deficit, or improvement in subjects receiving Compound 1: 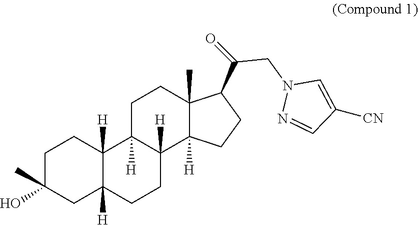
the method comprising:
administering a battery of Cogstate tests to the subject.

22. The method of claim 21, wherein the Cogstate tests comprise the Behavioral Pattern Separation Object test, the Continuous Paired Associate Learning test, the Detection test, the Face Name Associative Memory Exam, the Go-No Go test, the Groton Maze Learning Test, the Identification test, the International Shopping List Test, the One Back test, the One Card Learning test, the Set-Shifting test, the Social-Emotional Cognition Test, or the Two Back test.

23. The method of claim 21, wherein the Cogstate tests administered to the subject comprise the Detection test, the Identification test, the International Shopping List Test with Delayed Recall, and the Groton Maze Learning Test.

24. The method of claim 21, wherein the Cogstate tests are administered before administration of Compound 1.

25. The method of claim 21, wherein the Cogstate tests are administered during the administration of Compound 1.

26. The method of claim 21, wherein the Cogstate tests are administered after the administration of Compound 1.

27. The method of claim 21, wherein the Cogstate tests are administered before administration of Compound 1, during the administration of Compound 1, and after the administration of Compound 1.

28. The method of claim 21, wherein the method assesses the cognitive decline in the subject.

29. The method of claim 21, wherein the method assesses the cognitive deficit in the subject.

30. The method of claim 21, wherein the method assesses the cognitive improvement in the subject.

31. The method of claim 21, wherein the subject is a subject with depression or anxiety.

32. The method of claim 21, wherein the subject is a subject with major depressive disorder.
